(19) United States
(12) Patent Application Publication
(10) Pub. No.: US 2023/0293433 A1
(43) Pub. Date: Sep. 21, 2023

(54) METHOD OF MANUFACTURING MICELLES FOR DRUG DELIVERY

(71) Applicant: Wisconsin Alumni Research Foundation, Madison, WI (US)

(72) Inventors: Glen Kwon, Madison, WI (US); Moteza Rasoulianboroujeni, Madison, WI (US); Rae Hyung Kang, Madison, WI (US)

(21) Appl. No.: 18/122,661

(22) Filed: Mar. 16, 2023

Related U.S. Application Data
(60) Provisional application No. 63/321,064, filed on Mar. 17, 2022.

(51) Int. Cl. A61K 9/107; A61K 31/337; A61K 31/517; A61K 33/244

(52) U.S. Cl. CPC A61K 9/1075; A61K 31/337; A61K 31/517; A61K 33/244

(57) ABSTRACT

The present technology relates generally to a method of manufacture comprising: preparing at a first temperature a non-supersaturated solution comprising a micelle forming block copolymer and a drug in a polyethylene glycol (PEG) solvent; and diluting the non-supersaturated solution with water at a second temperature to form an aqueous solution comprising drug-loaded micelles without crystallization of the PEG block(s) of the block copolymer; wherein the block copolymer comprises at least one PEG block and a second biocompatible polymer block that is not PEG, the drug has a water solubility of about or less than 10 mg/mL at 25° C., and the second temperature is lower than the first temperature, but does not allow the cooled solution to become supersaturated.

### CROSS-REFERENCE TO RELATED APPLICATIONS

This application claims the benefit of and priority to U.S. Provisional Appl. No. 63/321,064, filed Mar. 17, 2022, the contents of which are incorporated herein by reference in their entirety for any and all purposes.

### GOVERNMENT RIGHTS

This invention was made with government support under 1R01CA257837-01, awarded by the National Institutes of Health. The government has certain rights in the invention.

### FIELD

The present disclosure relates generally to methods of manufacturing polymeric micelles for drug delivery. More specifically they relate to methods using a block copolymer and drug in a polyethylene glycol solvent, wherein the block copolymer comprises at least one poly(ethylene glycol) block (PEG) and a second biocompatible polymer block other than PEG, and the drug has a water solubility of about or less than 10 mg/mL at 25° C.

### BACKGROUND

Despite the potential advantages, a relatively small number of nanomedicine formulations have been approved for clinical use to date. The complex nature of nanomedicines compared with standard pharmaceuticals poses numerous challenges and obstacles in different stages of development. A reproducible scale-up and manufacturing process to obtain a consistent product with desired physicochemical and biological characteristics is important for any formulation, especially in the later stages of development. Scaling up from the laboratory to commercial production may result in discrepancies in the product performance. High endotoxin levels, residual solvent beyond the safety limit, poor reproducibility in the manufacturing process and substantial inter-batch variations in particle size are just some of the obstacles faced at larger scales of production. All appear to share one thing in common: fundamental differences with the genuine product resulting from differences in manufacturing Therefore, a potentially successful fabrication procedure needs to be scalable and comply with safety and regulatory challenges.

Polymeric micelles, as a drug carrier, are an important subcategory of nanomedicine which form as a result of self-assembly of a diblock or multiblock copolymer. Polymeric micelles are postulated to provide solubilization of water-insoluble or poorly soluble drugs, controlled drug release, and targeting among other functions in cancer treatment. The polymeric micelles are also significantly less toxic than typical anti-cancer drugs encapsulated within the polymeric micelles. In polymeric micelle formulations, polyethylene glycol (PEG) is often selected as the corona forming block due to its capability for reticuloendothelial system (RES) escape and prolonged circulation. Use of water as a solvent for PEG prompts the formation of micelles with hydrophobic cores for drug encapsulation and consequent solubilization. PEG-b-PLA is an example of micelle-forming block copolymer that does not disturb whole blood or its components and is compliant with the required standards for intravenous injection.

Existing methods of fabrication for drug-loaded polymeric micelles typically include the dissolution of both copolymer and drug in a non-selective organic solvent followed by addition of water as the selective solvent for one of the blocks to induce micellization. The latter step can be done either after complete removal of the organic solvent, such as in thin film hydration and freeze-drying methods, or in the presence of organic solvent to replace, emulsify or change the selectivity, such as in dialysis, emulsification-solvent evaporation or cosolvent azeotrope evaporation techniques, respectively. In the latter group of methods, the micelles form in the presence of organic solvent when a critical water concentration is reached, and the final product can be obtained by the complete removal of the organic solvent thereafter. The use of organic solvent, complex processes and specialty equipment in such fabrication routes leads to serious complications in bench-to-bedside translation. It includes but is not limited to technical, safety, environmental and economic challenges during the production as well as regulatory concerns about the final product due to residual solvent and reproducibility of nanoscale properties in large-scale batches.

### SUMMARY OF THE INVENTION

The present technology provides scalable methods for the production of drug-loaded polymeric micelles. The methods may be used, e.g., to produce micelles from block copolymers that comprise at least one PEG block and at least one poly(lactic acid) block such as PEG-b-PLA. The methods utilizes PEG as both a solvent for the block copolymer and as a lyoprotectant for the resulting micelles. The methods yield high encapsulation efficiency with a variety of different hydrophobic drugs and copolymers and deliver high drug loadings with reproducible particle size distributions. The resulting micelle formulations may be freeze-dried successfully without any significant change in encapsulation and particle size distribution. The present methods use no organic solvent, does not require a lyophilization aid (e.g., lactose, mannitol, sorbitol, or sucrose), does not require an ionizable salt (e.g., organic or inorganic), and are rapid, simple and cost-effective.

The methods include a method of manufacture comprising: preparing at a first temperature a non-supersaturated solution comprising a micelle-forming block copolymer and a drug in a polyethylene glycol (PEG) solvent; and diluting the non-supersaturated solution with water at a second temperature to form an aqueous solution comprising drug-loaded micelles without crystallization of the PEG block(s) of the block copolymer; wherein the block copolymer comprises at least one PEG block and a second biocompatible polymer block that is not PEG, the drug has a water solubility of about or less than 10 mg/mL at 25° C., and the second temperature is lower than the first temperature, but does not allow the cooled solution to become supersaturated.

### BRIEF DESCRIPTION OF THE DRAWINGS

FIGS. 1A-1C. FIG. 1A is a schematic phase diagram for PEG-b-PLA diblock copolymer mixed with liquid PEG. FIG. 1B shows formation of crystals from 5 wt. % PEG_{4kDa}-b-PLA_{2.2kDa} in liquid PEG mixture after 2 h incubation at 25° C. captured by light (top) and laser (bottom) microscopy. FIG. 1C shows graphs of isothermal crystallization kinetics for 5 wt. % PEG_{4kDa}-b-PLA_{2.2kDa} in liquid PEG mixture obtained by DSC at different isothermal hold temperatures (25° C., 30° C., 35° C., 40° C.).

FIGS. 2A-2D. FIG. 2A is a schematic representation of an illustrative embodiment of micelle preparation according to the present technology. The effect of PEG molecular weight and the mixture incubation temperature on (FIG. 2B) encapsulation efficiency (33 wt. % target loading) and (FIG. 2C) average particle size of the micelles. FIG. 2D shows encapsulation efficiency as a function of target drug loading when the micelles prepared using optimal parameters. Statistical analysis: n=6, * p<0.05, ** p<0.01, ***p<0.001, ****p<0.0001.

FIGS. 3A-3C. Graphs of encapsulation efficiency are shown for (FIG. 3A) rapid hydration of 10 wt. %, (FIG. 3B) slow hydration of 5 wt. % and (FIG. 3C) slow hydration of 10 wt. % PEG-b-PLA in the mixture for different molecular weights of PEG.

FIG. 4 shows a graph of PTX concentration in PEG-water mixture vs. water content in such mixture for different molecular weights of PEG

FIG. 5 shows a graph of scattered light intensity as a function of PEG-b-PLA concentration in PEG1000 in the absence or presence of water (3:1 w/w ratio). The onset of intensity jump was deemed as CMC.

FIGS. 6A-6F. FIG. 6A shows a micelle formation diagram for a PEG_{4kDa}-b-PLA_{2.2kDa}/PEG1000/water mixture at 40° C.; compositions within the upper right-hand region produce polymeric micelles; the boundary line between the two regions gives the approximate CMC for a specific water:PEG ratio or CWC for a specific initial copolymer concentration in PEG1000; the dashed and dotted boxes illustrate CMC at 3:1 w/w water:PEG ratio and CWC at 5.0 wt. % initial copolymer concentration, respectively. DLS correlograms for compositions (FIG. 6B) near CMC at 3:1 w/w water:PEG1000 ratio, (FIG. 6C) CWC at 5.0 wt. % initial copolymer concentration in PEG1000 and (FIG. 6D) CWC at 5.0 wt. % initial copolymer concentration in PEG1000 and 33 wt. % PTX target loading (“cp” stands for “copolymer”). FIG. 6E shows a schematic representation of the proposed mechanism of PEG-b-PLA micelle formation in an illustrative embodiment of the present method. FIG. 6F shows encapsulation efficiency of PTX when loaded in polymeric micelles by rapid mixing using acetonitrile (ACN) or PEG1000 as the solvent. Statistical analysis: n=6, *p<0.05, **p<0.01, *** p<0.001, **** p<0.0001.

FIG. 7 shows DLS correlograms for compositions near CWC at 5.0 wt. % initial copolymer concentration in PEG1000 at 60° C. (“cp” stands for “copolymer”).

FIGS. 8A-8D. FIG. 8A shows effect of incubation temperature on the particle size distribution for a PTX-free sample. FIG. 8B shows the particle size distribution for micelles obtained by hydration of the copolymer/PEG200-600 mixture incubated at 25° C. for different periods. FIG. 8C shows the effect of cooling rate to 25° C. on encapsulation efficiency. FIG. 8D shows the effect of prehydration at 40° C. followed by hydration at 25° C. on encapsulation efficiency. Statistical analysis: n=6, *p<0.05, **p<0.01, ***p<0.001, **** p<0.0001.

FIGS. 9A-9F. FIG. 9A shows the stability of PTX-loaded micelles at room temperature. FIG. 9B shows encapsulation and stability of 17AAG-loaded polymeric micelles at room temperature. FIG. 9C shows the effect of molecular weight of PEG and PLA blocks of PEG-b-PLA copolymer on the characteristics of the corresponding PTX-loaded micelles. FIG. 9D shows the effect of 25× scale-up on the characteristics of PTX-loaded micelles made by an illustrative embodiment of the present methods (also referred to as “PEG-assist”) using PEG_{4kDa}-b-PLA_{2kDa}. FIG. 9E shows the DSC thermogram of PEG_{4kDa}-b-PLA_{2kDa}/PEG1000/water mixture; heated at 10° C./min after getting frozen at the same rate. FIG. 9F shows the characteristics of PTX-loaded micelles before and after freeze-drying at −35° C. and 15 Pa for 72 h. Statistical analysis: n=6, *p<0.05, **p<0.01, *** p<0.001, **** p<0.0001.

FIGS. 10A-10B. The graphs show the average particle size change over time for (FIG. 10A) PTX and (FIG. 10B) 17AAG formulations prepared by an illustrative embodiment of the present methods at 20 wt. % drug loading and 1 mg/mL drug level.

FIG. 11 shows a schematic illustration of an illustrative embodiment of a method of the present technology using PEG_{4kDa}-b-PLA_{2kDa} to obtain freeze-dried samples.

FIGS. 12A-12B show plots of sample data for a block copolymer (P_{A}=90, P_{B}=30) with X_{AB}=0 χ_{AB}=0 and a small molecule solvent (Pₛ=1) when block copolymer A-B is mixed with the solvent at 5 wt. % concentration.

FIGS. 13A-13B show sample data for a block copolymer (P_{A}=90, P_{B}=30) with a different block copolymer (X_{AB}=0.2) and a small molecule solvent (P_{S}=1) P_{S}=1 when block copolymer A-B is mixed with the solvent at 5 wt. % concentration.

FIG. 14 displays the outcome of modeling (Example 4) for different X_{AB} χ_{AB} values ranging from 0-0.2 and different degrees of polymerization for solvent (P_{S}=1 to 5).

FIGS. 15A-15C are sample data plots shows how high χ_{AB} requires the application of higher degree of polymerization (P_{S}>20) of homopolymer/oligomer as the solvent to obtain micelles.

FIGS. 16A-16C show stability of different prodrug formulations (FIG. 16A: oLA₈-PTX; FIG. 16B: oLA₈-DTX; and FIG. 16C: oLA₈-RAP, respectively) made by PEG-assist method at room temperature. Percentage encapsulated prodrug was used as an indication of stability over time.

FIGS. 17A-17C show stability of different prodrug formulations (FIG. 17A: oLA₈-PTX; FIG. 17B: oLA₈-DTX; and FIG. 17C: oLA₈-RAP, respectively) made by PEG-assist method at room temperature. Particle size variation was used as an indication of stability over time.

FIGS. 18A-18D. FIG. 18A shows the concentration of individual prodrugs oLA₈-PTX, oLA₈-RAP and their combination oLA₈-PTX/oLA₈-RAP (5:1) together with parent drugs combination and Abraxane in rodent plasma upon injection. FIGS. 18B-18D show tumor volume (FIG. 18B), relative body weight (FIG. 18C), and survival rate (FIG. 18D) for individual oLA₈-PTX and oLA₈-RAP prodrugs and combination of PTX/RAP and oLA₈-PTX/oLA₈-RAP (5:1); saline and Abraxane were used as negative and positive controls, respectively, for in vivo anti-tumor efficacy.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the term “micelle-forming block copolymer” refers to any block copolymer that forms a micelle in aqueous solution at or above its critical micelle concentration.

The present technology provides pharmaceutical compositions and medicaments comprising any of one of the embodiments of the micelles including any of the drugs and/or drug conjugates disclosed herein and a pharmaceutically acceptable carrier or one or more excipients. The compositions may be used in the methods and treatments described herein. The pharmaceutical composition may include an effective amount of any of one of the embodiments of the compounds of the present technology disclosed herein. In any of the above embodiments, the effective amount may be determined in relation to a subject. “Effective amount” refers to the amount of a compound, conjugate, micelle or composition required to produce a desired effect. One example of an effective amount includes amounts or dosages that yield acceptable toxicity and bioavailability levels for therapeutic (pharmaceutical) use including, but not limited to, the treatment of cancers or cardiovascular disease such as restenosis.

As used herein, a “subject” or “patient” is a mammal, such as a cat, dog, rodent or primate. In any embodiments, the subject is a human, and, preferably, a human suffering from a cancer sensitive to any drug disclosed herein, i.e., a cancer capable of treatment with an effective amount of a drug disclosed herein, such as paclitaxel, docetaxel, rapamycin, everolimus, selumetinib, binimetinib, GDC-0623, or any other drug, derivative or conjugate described herein. The term “subject” and “patient” can be used interchangeably.

In one aspect, the present technology provides methods of manufacturing drug loaded micelles. The methods include preparing at a first temperature a non-supersaturated solution comprising a micelle-forming block copolymer and a drug in a polyethylene glycol (PEG) solvent; and diluting the non-supersaturated solution with water at a second temperature to form an aqueous solution comprising drug-loaded micelles without crystallization of PEG. The block copolymer includes at least one PEG block and a second polymer block that is not PEG, e.g., a poly(lactic acid) block. The drug may have a water solubility of about or less than 10 mg/mL at 25° C. In the methods, the second temperature is lower than the first temperature, but does not allow the cooled solution to become supersaturated.

A variety of PEG solvents may be used in the present methods. For example, the PEG solvent used in the present method may have a weight average molecular weight of about 200 Da to about 20 kDa, e.g., about 200, about 400, about 600, about 800, about 1,000, about 1,200, about 1,400, about 1,600, about 1,800, about 2,000, about 2,500, about 3,000, about 3,500, about 4,000, about 5,000, about 6,000, about 7,000, about 8,000, about 9,000, about 10,000, about 12,000, about 14,000, about 16,000, about 20,000 Da or a range between and including any two of the foregoing values, or a range above one of the foregoing values up to and including a second of the foregoing values. In any embodiments, the PEG solvent may have a weight average molecular weight of 600 Da to about 4, 6, or 20 kDa, or from greater than 600 to about 20 kDa, or about 800 Da to about 5, 4 or 2 kDa, or about 1 kDa, about 2 kDa or about 4 kDa. The PEG solvent need not be liquid at room temperature, as it may be heated until it forms a liquid and used as a liquid. The PEG solvent and temperature should be selected, however, so as to prevent the at least one PEG block of the micelle-forming block copolymer from crystallizing prior to or during the diluting step, and may further be selected to induce liquid-liquid phase separation between the block copolymer, water and the PEG solvent during/after the diluting step.

In the present methods, the non-supersaturated solution may be prepared in any of several ways. For example, preparing the non-supersaturated solution may include heating a mixture of the block copolymer, drug and the PEG solvent at the first temperature until the non-supersaturated solution forms. Alternatively, preparing the non-supersaturated solution may include heating a mixture of the block copolymer and the PEG solvent to form a solution or partial solution, adding the drug, and heating at the first temperature the mixture of block copolymer, drug, and PEG solvent to form the non-supersaturated solution. Preparing the non-supersaturated solution may instead include heating a mixture of the drug and the PEG solvent to form a solution or partial solution, adding the block copolymer, and heating at the first temperature the mixture of drug, block copolymer, and PEG solvent to form the non-supersaturated solution.

In the present method, the non-supersaturated solution may be equilibrated at the first temperature for at least 0.1 h after formation of the non-supersaturated solution. In any embodiment, the non-supersaturated solution may be equilibrated at the first temperature for 0.1 h to 10 h after formation of the non-supersaturated solution. For example, the non-supersaturated solution may be equilibrated at the first temperature for 0.1 h, 0.2 h, 0.3 h, 0.4 h, 0.5 h, 0.75 h, 1 h, 1.25 h, 1.5 h, 1.75 h, 2 h, 2.5, 3, h, 3.5 h, 4 h, 4.5 h, 5 h, 6 h, 7 h, 8 h, 9 h, 10 h or a period of time ranging between any two of the foregoing values, e.g., 0.3 h to 3 h.

In the present method, diluting the non-supersaturated solution with water may be done by any convenient method such as a single addition of water to the non-supersaturated solution or by two or more additions of the water to the non-supersaturated solution. In any embodiments the diluting step may include mixing the non-supersaturated solution and the water to homogeneity quickly, e.g., in less than an hour, less than 0.9 h, less than 0.8 h, less than 0.7 h, less than 0.6 h, less than 0.5 h, less than 0.4 h., less than 0.3 h, less than 0.2 h, or less than 0.1 h. In any embodiments, mixing the non-supersaturated solution and the water to homogeneity may be performed over the course of 1 second to less than an hour, e.g., from 1 second to less than less than 0.9 h, less than 0.8 h, less than 0.7 h, less than 0.6 h, less than 0.5 h, less than 0.4 h., less than 0.3 h, less than 0.2 h, or less than 0.1 h. In certain embodiments the mixing to homogeneity may be performed over 1 second, 2 seconds, 3 seconds, 4 seconds, 5 seconds, 10 seconds, 15 seconds, 20 seconds, 30 seconds, 40 seconds, 50 seconds, 1 minute, 1.5 minutes, 2 minutes, 3 minutes, 4 minutes, 5 minutes, 6 minutes or a range between any two of the foregoing. In any embodiments, the non-supersaturated solution is prepared continuously and diluted continuously with water to continuously form the aqueous solution.

In any embodiment of the present methods, the non-saturated solution may be an under-saturated solution at the first temperature. Thus, the first temperature may be selected such that non-supersaturated solution is under-saturated, i.e., the dissolved block copolymer and drug are below their saturation points in the particular PEG being used as a solvent. In any embodiments, the first temperature may be from 40° C. to 90° C., such as 40, 45, 50, 55, 60, 65, 70, 75, 80, 85 or 90° C., or a range between any two of the foregoing temperatures, e.g., from 50° C. to 70° C. In any embodiments the first temperature may be about 60° C.

In any embodiments, the present methods may further include cooling the non-supersaturated solution to the second temperature such that the solution remains non-supersaturated. In any embodiments of the present methods, the non-saturated solution may be a saturated solution at the second temperature. Thus, the second temperature may be selected such that the non-supersaturated solution is saturated, i.e., the dissolved block copolymer and drug are at one or both of their saturation points. In any embodiments the second temperature may be from 30° C. to 50° C., such as 30° C., 35° C., 40° C., 45° C., 50° C. or a range between any two of the foregoing temperatures, e.g., from 35° C. to 45° C. In any embodiments the second temperature may be about 40° C.

In any embodiment of the present methods, the non-saturated solution may be incubated at the second temperature for 0.1 h, 0.2 h, 0.3 h, 0.4 h, 0.5 h, 0.75 h, 1 h, 1.25 h, 1.5 h, 1.75 h, 2 h, 2.5, 3, h, 3.5 h, 4 h, 4.5 h, 5 h, 6 h, 7 h, 8 h, 9 h, 10 h or a period of time ranging between any two of the foregoing values, e.g., 0.5 h to 3 h.

The present methods may further include freeze-drying the aqueous solution comprising drug-loaded micelles to provide a freeze-dried powder. The freeze-dried powder may be reconstituted with water to provide a solution of drug-loaded micelles. It is an advantage of the present methods that the aqueous solution, lacking organic solvents (or too much PEG) that must be removed, may have a eutectic point above −50° C. Many aqueous/organic systems that have previously been used for micelle formation have eutectic points below −50° C., requiring removal of at least some of the organic solvent before the micelle solution may be freeze-dried. Thus, in any embodiments of the present methods, the PEG solvent need not be substantially removed from the aqueous solution. By substantially is meant at least 90% of the PEG is not removed from the aqueous solution, and typically even higher amounts of the PEG are not removed. In some embodiments, at least 95%, at least 98%, at least 99% or even 100% of the PEG is not removed but remains in the aqueous solution. Thus any technique that would be necessary to remove organic solvents (or too much PEG) is unnecessary to use in the present methods. For example, in the present methods, the aqueous solution may not be dialyzed.

In methods of the present technology, the block copolymer that forms the micelles includes any suitable di or tri-block copolymer having at least one PEG block and at least one other polymer block. The other polymer blocks may be selected from a poly(lactic acid) block, a polypropylene glycol (PPG) block, a poly(lactic-co-glycolic acid) block or a polycaprolactone block (PCL). For example, the block copolymer may be PEG-b-PLA (also known as PEG-PLA) or may be PEG-b-PLA-b-PEG. The poly(lactic acid) block can include (D-lactic acid), (L-lactic acid), (D,L-lactic acid), or combinations thereof. Various forms of PEG-b-PLA are available commercially, such as from Polymer Source, Inc., Montreal, Quebec, or Jenkem, Inc., or they can be prepared according to methods well known to those of skill in the art. The block copolymer may be a polaxamer, i.e. a triblock polymer having PEG-b-PPG-b-PEG. Such polaxamers are commercially available from, e.g., BASF as PLURONIC and KOLLIPHOR. The other polymer block may be selected from other hydrophobic polymers such as poly(propylene oxide), polystyrene, poly(methyl methacrylate) and the like. For example, the block copolymer may be poly(ethylene glycol)-block-poly(propylene oxide) (PEG-PPO), poly(ethylene glycol)-block-polystyrene (PEG-PS), poly(ethylene glycol)-block-poly(methyl methacrylate) (PEG-PMMA).

In another aspect, one or both PEG blocks in the block copolymer may be replaced by other hydrophilic polymers. Thus, in place of or in addition to PEG, the hydrophilic block of the block copolymer may be selected from poly(vinyl alcohol) (PVOH), poly(2-alkyl/aryl-2-oxazoline)s (commonly abbreviated as PAOx, POx, or POZ) and the like. For example, the block copolymer may be poly(vinyl alcohol)-block-poly-ε-caprolactone (PVOH-PCL), poly(vinyl alcohol)-block-poly(lactic-co-glycolic acid) (PVOH-PLGA), poly(vinyl alcohol)-block-poly(propylene oxide) (PVOH-PPO), poly(vinyl alcohol)-block-polystyrene (PVOH-PS), poly(vinyl alcohol)-block-(methyl methacrylate) (PVOH-PMMA), poly(vinyl alcohol)-block-polypropylene (PVOH-PP). In this case, PEG or an oligomer/homopolymers with the same repeating unit as the hydrophilic block of the block copolymer can be used as the solvent.

The molecular weight of the poly(ethylene glycol) block in the block copolymer which forms the micelles can be about 1,000 to about 35,000 g/mol, or any increment of about 500 g/mol within said range. (All polymer molecular weights referred to herein will be understood to be weight average molecular weights.) For example, the molecular weight of the PEG block may be 1,000, 1,500, 2,000, 2,500, 3,000, 3,500, 4,000, 4,500, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000 or a range between and including any two of the foregoing values.

Suitable blocks of the poly(lactic acid) for the micelle-forming block copolymer can have molecular weights of about 1,000 to about 15,000 g/mol, or any increment of about 500 g/mol within said range. For example, the molecular weight of the PLA block may be 1,000, 1,500, 2,000, 2,500, 3,000, 3,500, 4,000, 4,500, 5,000, 6,000, 6,500, 7,000, 7,5000, 8,000, 8,500, 9,000, 9,500, 10,000, 10,500, 11,000, 11,500, 12,000, 12,500, 13,000, 13,500, 14,000, 14,500, 15,000, or a range between and including any two of the foregoing values. The PEG block can terminate in an alkyl group, e.g., a C1-6 alkyl group, such as a methyl group (e.g., a methoxy ether) or any suitable protecting, capping, or blocking group, including without limitation OH, NH₂, urethane, SH, COOH, biotin, a dye or a targeting ligand. In some embodiments, the PEG block is terminated in a targeting ligand (e.g., a PSMA (prostate-specific membrane-antigen) targeting ligand such as ACUPA). In some embodiments, the molecular weight of the poly(ethylene glycol) block of PEG-b-PLA is about 1,000 to about 35,000 g/mol and the molecular weight of the poly(lactic acid) block of PEG-b-PLA is about 1,000 to about 15,000 g/mol. In some embodiments, the molecular weight of the poly(ethylene glycol) block is about 1,500 to about 14,000 g/mol, and the molecular weight of the poly(lactic acid) block is about 1,500 to about 7,000 g/mol.

The phrase “targeting ligand” refers to a ligand that binds to “a targeted receptor” that distinguishes the cell being targeted from other cells. The ligands may be capable of binding due to expression or preferential expression of a receptor for the ligand, accessible for ligand binding, on the target cells. Examples of such ligands include PSMA (prostate-specific membrane-antigen) targeting ligands (e.g., ACUPA), GE11 peptide, anti-EGFR nanobody, RGD, RGD containing peptides, cRGD ((cyclo(RGDfC)), KE108 peptide, octreotide, folic acid, prostate-specific membrane antigen (PSMA) aptamer, TRC105, a human/murine chimeric IgG1 monoclonal antibody, mannose, and cholera toxin B (CTB), glucose, amino acids, all-trans retinoic acid, RVG peptide (YTIWMPENPRPGTPCDIFTNSRGKRASNG), N-acetylgalactosamine (GalNAc), mannitol, hyaluronic acid, Rituximab, Trastuzumab, Bevacizumab, Alemtuzumab, Panitumumab, RGD, DARPins, RNA aptamers, DNA aptamers. Additional examples of such ligands include peptide ligands identified from library screens, monoclonal or polyclonal antibodies, Fab or scFv (i.e., a single chain variable region) fragments of antibodies, growth factors, such as EGF, FGF, insulin, and insulin-like growth factors, and homologous polypeptides, somatostatin and its analogs, transferrin, lipoprotein complexes, bile salts, selecting, steroid hormones, microtubule-associated sequence (MTAS), various galectins, δ-opioid receptor ligands, cholecystokinin A receptor ligands, ligands specific for angiotensin AT1 or AT2 receptors, peroxisome proliferator-activated receptor y ligands, β-lactam antibiotics, small organic molecules including antimicrobial drugs, and other molecules that bind specifically to a receptor preferentially expressed on the surface of targeted cells, or fragments of any of these molecules. In some embodiments, the targeting ligand is ACUPA.

The phrase “a targeted receptor” refers to a receptor expressed by a cell that is capable of binding a cell targeting ligand. The receptor may be expressed on the surface of the cell. The receptor may be a transmembrane receptor. Examples of such targeted receptors include EGFR, integrin, somatostatin receptor, folate receptor, prostate-specific membrane antigen, CD105, mannose receptor, estrogen receptor, GLUT1, LAT1, nicotinic acetylcholine receptors (nAChR), asialoglycoprotein receptor, and GM1 ganglioside.

The present methods of manufacturing micelles may use PEG-b-PLA polymers of a variety of block sizes (e.g., a block size within a range described above) and in a variety of ratios of sizes. For example, the PEG:PLA ratio may be about 1:10 to about 10:1, or any integer ratio within said range, including without limitation 1:5, 1:3, 1:2, 1:1, 2:3, 2:1, 3:1, and 5:1. For example, weight average molecular weights (Ma) of the PEG-PLA polymers can include, but are not limited to, 2K-2K, 2K-3K, 3K-5K, 4K-2K, 5K-3K, 5K-6K, 6K-5K, 6K-6K, 8K-4K, 4K-8K, 12K-3K, 3K-12K, 12K-6K, 6K-12K (PEG-PLA, respectively) or a range between and including any two of the foregoing values.

One suitable block copolymer is a PEG-PLA that includes blocks of about 1-10 kDa (e.g., about 4 kDa (4K) and about 2 kDa (2K)) at an approximate 50:50 ratio. Use of this block copolymer resulted in high levels of drug loading in the micelles. Further specific examples of PEG-PLA molecular weights include 4.2K-b-1.9K; 5K-b-10K; 12K-b-6K; 2K-b-1.8K, and those described in the Examples below. Other suitable amphiphilic block copolymers that may be used are described in U.S. Pat. No. 4,745,160 (Churchill et al.) and U.S. Pat. No. 6,322,805 (Kim et al.). The drug-to-polymer ratio may be about 1:20 to about 2:1, or any integer ratio within said range. Specific examples of suitable drug-polymer ratios include, but are not limited to, about 2:1, about 3:2, about 1.2:1, about 1:1, about 3:5, about 2:5, about 1:2, about 1:5; about 1:7.5; about 1:10, about 1:20 or a range between and including any of the foregoing values.

In methods of the present technology, various amounts of block copolymer may be used to form the micelles. In any embodiments, the non-saturated solution may include 0.1 wt % to 50 wt % block copolymer. For example, the non-saturated solution may include 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 25 wt %, 30 wt %, 45 wt %, or 50 wt % block copolymer or a range between and including any two of the foregoing values. Thus, in further examples of the methods the non-saturated solution may include 1 wt % to 10 wt % block copolymer.

In the present methods, various amounts of the drug may be used to prepare the drug-loaded micelles. In any embodiments, the non-saturated solution may include 0.005 wt % to 50 wt % drug. For example, the non-saturated solution may include 0.005 wt %, 0.01 wt %, 0.05 wt %, 0.1 wt %, 0.2 wt %, 0.3 wt %, 0.4 wt %, 0.5 wt %, 0.75 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 9 wt %, 10 wt %, 11 wt %, 12, wt % 13 wt %, 14 wt %, 15 wt %, 16 wt %, 17 wt %, 18 wt %, 19 wt %, 20 wt %, 25 wt %, 30 wt %, 35 wt %, 40 wt %, 45 wt %, 50 wt % or a range between and including any two of the foregoing values. For example, the non-saturated solution may include 0.05 wt % to 5 wt % or to 10 wt % drug.

The present methods may be used to prepare drug-loaded polymeric micelles in which the drug has limited water solubility, e.g., less than 10 mg/mL, less than 5 mg/mL, or less than 1 mg/mL in water at 25° C. Examples of drugs that may be used in the present methods include but are not limited to those selected from the group consisting of paclitaxel, docetaxel, cabazitaxel, rapamycin, everolimus, selumetinib, binimetinib, GDC-0623, ICU 189150, doxorubicin, etoposide, 17-AAG, bicalutamide, embelin, suberoylanilide hydroxamic acid, β-lapachone, pifithrin-μ, sagopilone, thiocoraline, ABT-263, podophyllotoxin, simvastatin, efavirenz, platin-based prodrugs, VE-822, AZD5363, teniposide, AZD8055, rutin, PROTAC (e.g., Gefitinib-based PROTAC3), cerium oxide nanoparticles, derivatives and/or conjugates of any of the foregoing, and combinations of two or more thereof.

Paclitaxel (also referred to as “PTX”), docetaxel (also referred to as “DTX”), rapamycin (also referred to as “RAP”), and selumetinib are potent chemotherapeutic agents useful in the treatment of a variety of cancers and have the structures shown below.

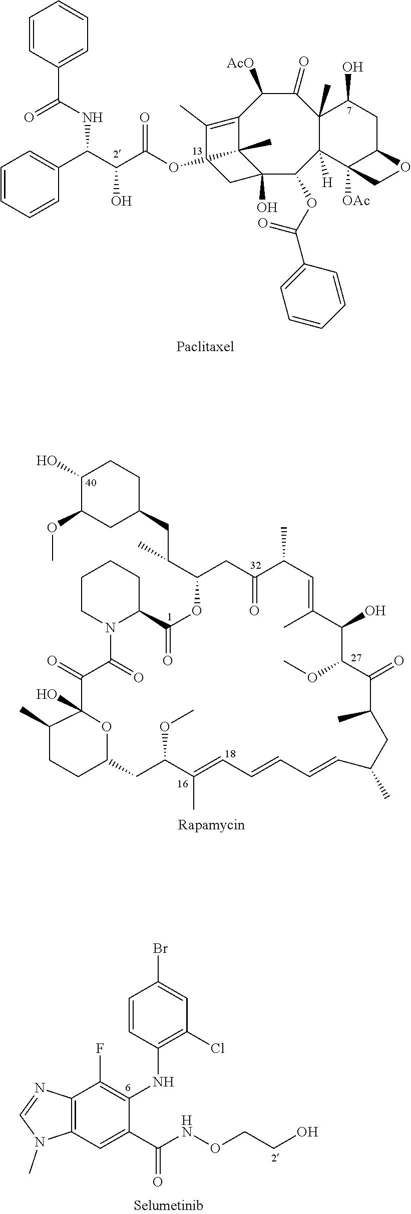

Derivatives of such drugs refer to analogs which retain most (e.g., >90% on a per carbon atom basis) or all of the carbon skeleton of the drug, but includes at least one modification, e.g., on a side chain. Such derivatives also retain similar but not necessarily identical activity. Thus, as used herein, a “paclitaxel derivative” is a compound that retains the carbocyclic/oxetane skeleton of paclitaxel (i.e., the taxane skeleton) but contains at least one modified side chain. In any embodiments, the modified side chain is other than the 7-hydroxyl. Paclitaxel derivatives of the present technology exhibit anti-cancer activity. For example, docetaxel is a paclitaxel derivative which contains a modification of the C-13 sidechain in which t-butyloxycarbonylamino replaces benzamido at the 3′-position. Other paclitaxel derivatives are known to those of skill in the art and include but are not limited to those described in Farina, V., “The chemistry and pharmacology of Taxol and its derivatives,” Elsevier, New York, 1995 (incorporated herein by reference in its entirety and for all purposes).

As used herein, a “rapamycin derivative” or “rapalog” is a compound that retains the macrocyclic lactone ring of rapamycin, but contains at least one modified side chain while retaining a free hydroxyl group on the C-40 position or a free hydroxyl attached to a modified side chain bonded to the C-40 position (e.g., everolimus). Rapamycin derivatives of the present technology exhibit anti-cancer activity. For example, everolimus is a rapamycin derivative with the structure below. Other rapamycin derivatives are known to those of skill in the art and include but are not limited to those described in Wander, S., et al., “Next-generation mTOR inhibitors in clinical oncology: how pathway complexity informs therapeutic strategy,” J. Clin. Invest., 121(4), 1231-1241 (2011) (incorporated herein by reference).

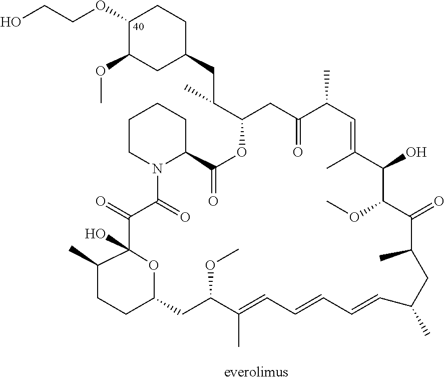 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

As used herein, a “selumetinib derivative” is a compound that retains the 6,5-fused ring system of selumetinib, but contains at least one modified side chain while retaining a free hydroxyl group on the C-2′ position (e.g., binimetinib, GDC-0623, and ARRY-300). Selumetinib derivatives of the present technology exhibit anti-cancer activity. For example, binimetinib and GDC-0623 are selumetinib derivatives with the structures below. Other selumetinib derivatives are known to those of skill in the art and include but are not limited to those described in Jokinen, E., et al., “MEK and PI3K inhibition in solid tumors: rationale and evidence to date,” Ther. Adv. Med. Oncol., 7(3), 170-180 (2015) (incorporated herein by reference).

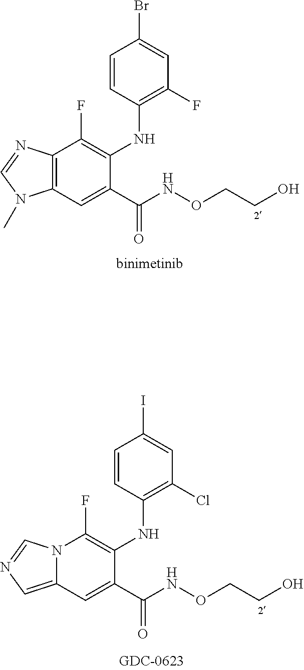 [SMILES: Cn1cnc2c(F)c(Nc3ccc(Br)cc3F)c(C(=O)NOCCO)cc21 ; O=C(NOCCO)c1cc2cncn2c(F)c1Nc1ccc(I)cc1Cl]

PROTAC (proteolysis targeting chimera) is a heterobifunctional molecule composed of two active domains and a linker, capable of removing specific unwanted proteins. In some embodiments, the PROTAC described herein is Gefitinib-based PROTAC3.

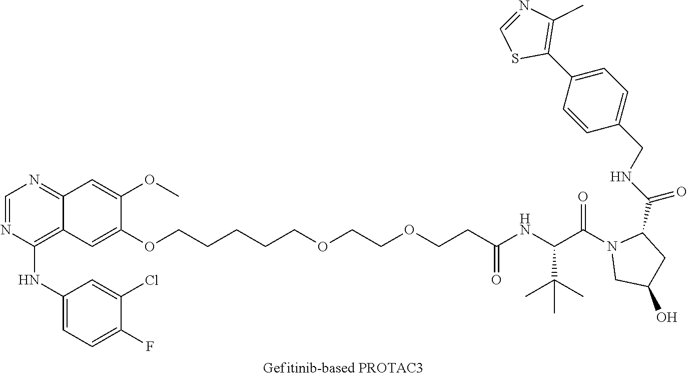 [SMILES: COc1cc2ncnc(Nc3ccc(F)c(Cl)c3)c2cc1OCCCCCOCCOCCC(=O)N[C@H](C(=O)N1C[C@H](O)C[C@H]1C(=O)NCc1ccc(-c2scnc2C)cc1)C(C)(C)C]

In any embodiments, the drug may be an oligolactic acid conjugate. Such conjugates are known in the art and described in, e.g., WO2017/158499 and WO2019/195164, the entire contents of which are incorporated herein by reference and for all purposes. In any embodiments, the drug conjugate may be 7-oligolactic acid conjugate of paclitaxel or a paclitaxel derivative (e.g., docetaxel) wherein the oligolactic acid comprises 2 to 24 lactic acid subunits (e.g., 8 lactic acid subunits, also referred to as “oLA₈”) and is attached through an ester linkage to the oxygen of the 7-hydroxyl of the paclitaxel or paclitaxel derivative (e.g., docetaxel); a 40-oligolactic acid conjugate of rapamycin or a rapamycin derivative wherein the oligolactic acid comprises 2 to 24 lactic acid subunits (e.g., 8 lactic acid subunits) and is attached through an ester linkage to the oxygen of the 40-hydroxyl of the rapamycin or rapamycin derivative; and/or a 2′-oligolactic acid conjugate of selumetinib or a selumetinib derivative wherein the oligolactic acid comprises 2 to 24 lactic acid subunits and is attached through an ester linkage to the oxygen of the 2′-hydroxyl of the selumetinib or selumetinib derivative.

The nanoparticles (e.g., cerium oxide nanoparticles) described herein may have an average particle size of about 1 nm to about 30 nm, including about 1 nm, about 5 nm, about 10 nm, about 15 nm, about 20 nm, about 25 nm, about 30 nm, or a range between and including any two of the foregoing values. In some embodiments, the nanoparticles (e.g., cerium oxide nanoparticles) described herein have an average particle size of less than 5 nm (e.g., about 3 nm to about 5 nm).

In another aspect, the present technology provides aqueous and freeze-dried compositions of micelles formed from PEG; block copolymers as described herein, and a 2- or 3-drug combination of a free paclitaxel/paclitaxel derivative, a free rapamycin/rapamycin derivative, and a free selumetinib/selumetinib derivative, or conjugates of any of the foregoing. As used herein, the term “free” refers to the unconjugated drug/drug derivatives. Such micelles may be more stable than the corresponding micelles individually loaded with free paclitaxel/paclitaxel derivatives, free rapamycin/rapamycin derivatives, or free selumetinib/selumetinib derivatives, or conjugates thereof.

Micelles of the present technology may be loaded with a wide range of amounts, including high amounts, of the drugs described herein. For example, the loading of the conjugates may be from about 2 wt % to about 60 wt % with respect to the mass of the micelles. Examples of conjugate loading in the micelles include about 3 wt %, about 4 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, or about 60 wt % with respect to the mass of the micelles, or a range between and including any two of the foregoing values. Thus, in some embodiments, the loading of the drugs range from about 5 wt % to about 40 wt %.

Loading of each drug in the micelles may also be expressed in terms of concentration. For example, the concentration of each drug may be from about 0.5 mg/mL to about 20 mg/mL with respect to the volume of the water in the composition. Examples of each drug concentration that may be obtained with the present technology include about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, about 6 mg/mL, about 8 mg/mL, about 10 mg/mL, about 12 mg/mL, about 14 mg/mL, about 16 mg/mL, about 18 mg/mL, or about 20 mg/mL with respect to the volume of the water in the composition, or a range between and including any two of the foregoing values. In any embodiments, the concentration of the drug may be about 0.8 to about 10 mg/mL.

The loading of each conjugate in the micelles may also be expressed in terms of encapsulation efficiency, that is the percentage of the initial drug which is actually incorporated into the micelles, and the loading target efficiency, the percentage of the initial amount of the drug with respect to the sum of the weight of the micelles and the weight of the initial amount of drug. For example, the encapsulation efficiency of each drug may be from about 50% to about 100% with respect to the initial mass of the drug. Examples of encapsulation efficiency include 50%, 55%, 60%, 65%, 70%, 75%, 80%, 85%, 90%, 95% and 100% or ranges between and including any two of the foregoing values. For example, encapsulation efficiency may be about 80% to about 100%. The loading target efficiency may be, e.g., between about 5% and about 60%. Examples of loading target efficiency in the micelles include about 5%, about 10%, about 15%, about 20%, about 25%, about 30%, about 35%, about 40%, about 45%, about 50%, or about 60% with respect to the sum of the mass of the micelles and the initial drug amount, or a range between and including any two of the foregoing values. Thus in any embodiments, the target loading efficiency of the drug in the micelles ranges from about 5% to about 35% or 40%.

Once prepared, the micelle-conjugate or micelle-drug compositions can be stored for extended periods of time under refrigeration, preferably at a temperature below about 5° C. Temperatures between about −20° C. and about 4° C. have been found to be suitable conditions for storage of most micelle-conjugate and micelle-drug compositions. For example, aqueous solutions of the present conjugate-loaded micelles may be stored at about 4° C. Freeze-dried micelle compositions as described herein can be stored at room temperature (for short periods such as 1, 2, 3, 4, 5, 6, 7, 8, 9, or 10 days or a range between and including any two of the foregoing values) or below and may be stored at −20° C. for prolonged periods (e.g., 1, 2, 3, or more years) and then rehydrated. Use of brown glass vials or other opaque containers to protect the micelle compositions from light can further extend effective lifetimes of the compositions.

In another aspect, the present technology provides methods of inhibiting or killing cancer cells sensitive to paclitaxel or a paclitaxel derivative, rapamycin or a rapamycin derivative, and/or selumetinib or a selumetinib derivative comprising contacting the cells with an effective inhibitory or lethal amount of any of the compositions described herein. In some such methods, the contacting is performed in vitro or in vivo. There are also provided methods of treatment including administering to a mammal suffering from a cancer sensitive to paclitaxel or a paclitaxel derivative, rapamycin or a rapamycin derivative, and/or selumetinib or a selumetinib derivative, an effective amount of any of the micelle compositions described herein. Examples of paclitaxel-sensitive, rapamycin-sensitive, and selumetinib-sensitive cancers include brain tumors, breast cancer, colon cancer, head and neck cancer, lung cancer, lymphoma, melanoma, neuroblastoma, ovarian cancer, pancreatic cancer, prostate cancer, angiosarcoma, and leukemia. In some embodiments, the cancer is breast cancer or lung cancer. In some embodiments, the effective amounts of two or three drug/drug derivative or drug/drug derivative conjugate as disclosed herein are synergistic, e.g., they have a more than additive effect or produce effects that cannot produced by a drug or drug conjugate alone.

In any of the embodiments of the present technology described herein, the pharmaceutical composition may be packaged in unit dosage form. The unit dosage form is effective in treating a cancer or restenosis. Generally, a unit dosage including a composition of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

Micelle compositions containing drugs, drug derivatives and/or conjugates thereof may be prepared as described herein and used to treat cancers and other diseases. The conjugates and compositions described herein may be used to prepare formulations and medicaments that treat restenosis or a cancer, such as leukemia, angiosarcoma, breast cancer, colorectal cancer, prostate cancer, lung cancer, brain cancer (such as gliomas), adenocarcinomas, or hepatomas. Such compositions can be in the form of, for example, granules, powders, tablets, capsules, syrup, suppositories, injections, emulsions, elixirs, suspensions or solutions. The instant compositions can be formulated for various routes of administration, for example, by parenteral, rectal, nasal, vaginal administration, or via implanted matrix or reservoir, or for restenosis, by drug-coated stent or balloon-based delivery. Parenteral or systemic administration includes, but is not limited to, subcutaneous, intravenous, intraperitoneal, and intramuscular, injections. The following dosage forms are given by way of example and should not be construed as limiting the instant present technology.

Injectable dosage forms generally include solutions or aqueous suspensions or oil in water suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution.

For injection, the pharmaceutical formulation and/or medicament may be a film or powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. In some embodiments, the injectable formulations include an isotonicity agent (e.g., NaCl and/or dextrose), buffer (e.g., phosphate) and/or a preservative.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The examples herein are provided to illustrate advantages of the present technology and to further assist a person of ordinary skill in the art with preparing micelle compositions of the present technology, pharmaceutical compositions, derivatives, metabolites, prodrugs, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

### Materials.

Polyethylene glycol-b-poly(D, L-lactide) (PEG-b-PLA, PEG block Mw: 4 kDa, PDLLA block Mw: 2.2 kDa) diblock copolymer was purchased from JenKem Technology (Plano, TX). PEG-b-PLA copolymer with different molecular weights were purchased from Advanced Polymer Materials Inc. (Quebec, Canada). PEGs of different molecular weights (i.e. 200, 400, 600 and 1000 Da) were purchased from Sigma-Aldrich (St. Louis, MO). Paclitaxel (PTX) was obtained from LC Laboratories (Woburn, MA). All other chemicals were of reagent grade.

### Crystallization—Differential Scanning Calorimetry (DSC)

PEG_{4kDa}-b-PLA_{2.2kDa} diblock copolymer granules (5.0 wt. %) were added to liquid PEG (Mw 200-600 Da), heated to 60° C. and vortexed to obtain a transparent mixture. After 30 min incubation, one drop of each solution (ca. 20 mg) was transferred to aluminum crucibles and allowed to cool down to room temperature prior to DSC analysis (DSC 404 F1 Pegasus, NETZSCH, Germany). The samples were heated to 80° C. and kept at this temperature for 15 min in order to erase thermal history in the first scan. The samples were then cooled down to different temperatures ranging from 25 to 40° C. for various periods ranging from 0.5 to 48 hours to induce isothermal crystallization. Heating the samples to 80° C. in the second scan yielded the melting onset temperature and enthalpy. A heating/cooling rate of 5° C./min was used for all the scans. The maximum crystallizable content (100%) was obtained by incubating samples at the corresponding temperature for 10 days followed by DSC scan using the same parameters.

### Micelle Preparation Protocol

Polymeric micelles were obtained by hydration of the PEG-b-PLA diblock copolymer/PEG mixture at different temperatures. Briefly, a transparent mixture (5.0 wt. % PEG-b-PLA in PEGs of different Mw) was obtained by heating the mixture to 60° C. in borosilicate glass vials, vortexing and incubation at the same temperature for 30 min. The transparent mixture was kept at 60° C. or cooled down to 40 or 25° C., incubated for 2 h and hydrated by addition of DI water with the same temperature.

In order to prepare drug-loaded micelles, PTX (at a weight ratio of 1:2 w/w PTX: PEG_{4kDa}-b-PLA_{2.2kDa} corresponding to target loading of ca. 33 wt. %) was added to the mixture prior to heating to 60° C. The amount of water when PTX was present in the formulation was adjusted to aim for 4 mg/mL PTX level. Such a high target loading and PTX level was used for optimization of preparation parameters only. The hydration process was performed under different conditions including rapid hydration (vortexing after addition of water) or slow hydration (a two-step hydration protocol consisting of addition of half of the target water volume followed by mixing and then addition of the rest followed by gentle mixing at 50 rpm in a rotary mixer). Micelles with the target loadings of 10 and 20 wt. % as well as 1 mg/mL PTX target level were prepared after optimizing the process parameters and used for other experiments such as stability measurement, scalability study and freeze-drying. To prepare formulations with 1 mg/mL PTX target level at 10 or 20 wt. % target loading, 1 mg PTX together with 9 or 4 mg copolymer were mixed with 170 or 75 mg PEG, respectively, and heated to 60° C. to obtain a transparent mixture. The added amounts of PEG keep the block copolymer concentration at 5.0 wt. % in the mixture. The mixture was cooled down to 40° C., incubated for 2 h followed by hydration with 1 mL DI water at 40° C. to obtain drug-loaded polymeric micelles. The samples were cooled down to room temperature and analyzed.

### Micelle Formation and Particle Size Analysis

Dynamic light scattering (DLS, nano ZS, Malvern) was used to measure particle size distribution and to investigate micelle formation. For particle size analysis, the measurements were carried out at 25° C. using water as dispersant.

For the micelle formation study, DLS was carried out for all the samples at two different temperatures (60 and 40° C.). The intensity of scattered light detected at 173° was recorded for different concentrations of PEG-b-PLA in PEG1000 ranging from 0.0 to 5.0 wt. % in the absence or presence of water (water/PEG weight ratio ranging from 0 to 3). The measured value for “count rate”, which represents the intensity of scattered light, is independent of the input values for viscosity, refractive index (RI) and dielectric constant of the solvent (the values of which are shown for PEG1000 in Table 1 at various temperatures.

TABLE 1
Properties of PEG1000 at various temperatures.

| | | | Refractive | Dielectric |
|---|---|---|---|---|
| PEG Mw | Temperature | Viscosity | index | constant |
| PEG1000 | 60 | 50.85 | 1.454755 | 10.74 |
| PEG1000 | 50 | 72.16 | 1.458544 | 11.15 |
| PEG1000 | 40 | 105.45 | 1.462419 | 11.55 |

Micelle formation results in a considerable increase in the intensity of scattered light and enhanced signal to noise ratio in autocorrelation function. According to Topel et al. [Topel, O., et al., “Determination of critical micelle concentration of polybutadiene-block-poly (ethyleneoxide) diblock copolymer by fluorescence spectroscopy and dynamic light scattering” J. Molecular Liquids, 2013. 177:40-43], DLS is as sensitive as fluorescence spectroscopy using pyrene in determining the critical micelle concentration (CMC) of a copolymer. A parameter named “Relative Scattered Light (RSL)” was defined as the ratio of “scattered light intensity by sample” to “scattered light intensity by dispersant” in order to minimize the effect of dispersion medium (water/PEG mixture):

$$Relative Scattered Light ( R S L ) = \frac{Derived Count Rate for sample}{Derived Count Rate for its dispersant} \qquad eq . 1$$

The onset of RSL increase can be considered as the onset of micelle formation, which in turn gives CMC and critical water concentration (CWC). DLS correlograms (correlation coefficient vs. time) were used to study micelle formation near the critical concentrations. Correlation coefficient indicates how fast a scattered light signal originated from certain entities in the sample decay by correlating the signal intensity with itself at very short intervals. Perfect and no correlation between the two signals are indicated by unity and zero, respectively. The signal decay (i.e. reduction of correlation coefficient from 1 to 0) is due to Brownian motion of the particles and is correlated with both particle size and medium viscosity.

### Encapsulation Efficiency

To measure the encapsulation efficiency, unencapsulated drug was first removed by centrifugation at 10000 g for 10 min. Following centrifugation, 50 μl of supernatant for each sample was mixed with 50 μl DI water and 400 μl acetonitrile (ACN). High performance liquid chromatography (HPLC, Prominence, Shimadzu, Japan) equipped with an autosampler, a C18 column and a UV-Vis detector was employed to measure PTX content. PTX was detected at 228 nm absorption wavelength by injecting 10 μl sample through an 80/20 ACN/water v/v mixture as the mobile phase under the flow rate of 0.5 mL/min. Encapsulation efficiency was defined as the weight ratio of encapsulated drug to the initial drug used. The target drug loading was defined as:

$$target drug loading ( wt . \% ) = 100 \times \frac{initial drug weight}{initial drug + copolymer weight} \qquad eq . 2$$

### Freeze-Drying

For freeze-drying, first, the melting onset of eutectic mixture was determined using DSC. The PEG_{4kDa}-b-PLA_{2kDa}/PEG1000/water mixture was cooled down to −80° C. at 10° C./min. The frozen mixture was then heated to room temperature at 10° C./min to detect the eutectic mixture and ice crystals melting transitions. The sample composition for freeze drying was 1 mg PTX, 4 mg copolymer, 75 mg PEG1000 and 1 mL water in a 3.7 mL standard glass vial. The freezing step was performed at −80° C. overnight. The freeze-dryer shelf temperature, based on the collected data, was set at −35° C. and freeze-drying was performed in a VirTis Advantage Pro freeze-dryer at 15 Pa for 72 h.

### Statistical Analysis

All the measurements are reported as Mean±SEM. One-way or two-way ANOVA were used to analyze statistical differences between different groups followed by Tukey's HSD or sidak's test as multiple comparison tests. The differences were considered significant if p<0.05.

### Example 1—Degree of Saturation and PEG Molecular Weight Influence the Physicochemical Properties of Drug-Loaded Polymeric Micelles

Diblock copolymers self-assemble into polymeric micelles in a selective environment for one of the blocks. Formation of PEG-b-PLA micelles in water, for example, is due to its selectivity for the PEG block. Therefore, dissolving the copolymer in a non-selective solvent (e.g. acetonitrile) followed by replacement with or addition of a selective solvent (e.g. water) constructs the basis of most fabrication methods for polymeric micelles. Addition of drugs to the initial solution results in drug encapsulation during self-assembly. We aimed to use PEG instead of organic solvents as the non-selective solvent in the preparation procedure. To begin with, we studied the solubility of PEG-b-PLA in liquid PEG.

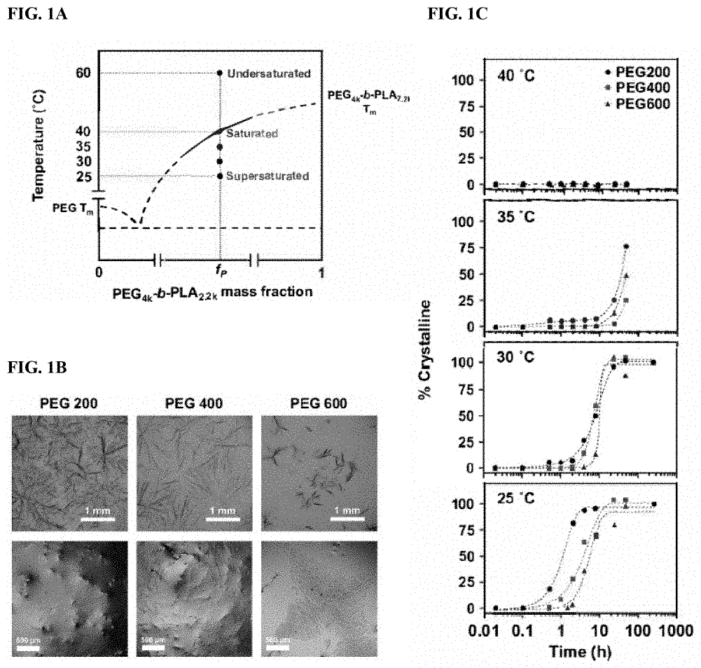

At elevated temperatures, mixing PEG_{4kDa}-b-PLA_{2.2kDa} with PEG of different molecular weights at 5.0 wt. % yields a transparent solution. Cooling this mixture to room temperature causes turbidity over time and results in a waxy solid. We assumed that this phenomenon is a result of crystallization of the PEG block of the copolymer at reduced temperatures. Therefore, PEG-b-PLA solubility in PEG is temperature dependent. In this case, the mixture is undersaturated at 60° C. and supersaturated at 25° C. (see FIGS. 1A-1C). We assessed the approximate saturation temperature for the mentioned composition by monitoring crystal formation at different temperatures over time using DSC. No crystals could be detected after 48 hours of isothermal hold at 40° C. and this temperature was supposed as approximate saturation temperature (FIGS. 1A-1C). We concluded that 5.0 wt. % PEG_{4kDa}-b-PLA_{2.2kDa} in liquid PEG is undersaturated at 60° C., saturated at 40° C. and supersaturated at 25° C.

Since the solubility of PEG-b-PLA in liquid PEG is considerably temperature-dependent, we examined the effect of temperature on encapsulation efficiency and particle size of PTX-loaded micelles obtained using PEG_{4kDa}-b-PLA_{2.2kDa}/PTX/liquid PEG. We selected 25, 40 or 60° C. to characterize supersaturated, saturated and undersaturated states of the solution, respectively. Addition of water to PEG-b-PLA/PTX/liquid PEG leads to formation of polymeric micelles by increasing the selectivity of the environment for the PEG block of the copolymer. In addition to temperature, we investigated the effect of PEG molecular weight as it may influence the solubilization power of the environment throughout the self-assembly process.

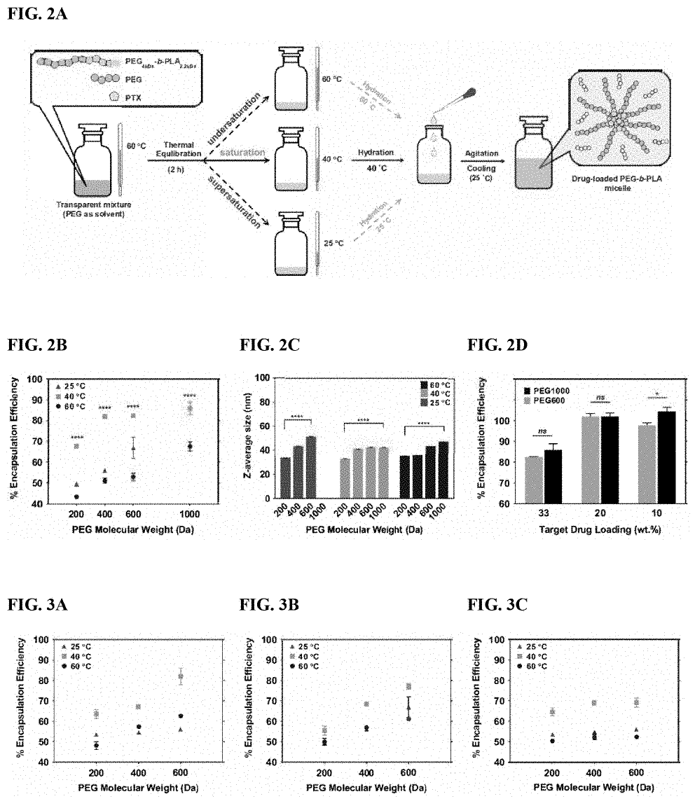

We incubated the copolymer/PTX/PEG (5.0:2.5:92.5 wt. %) for 2 h at 25, 40 or 60° C. The micelles were obtained by rapid hydration of the mixture at the same temperature (FIG. 2A). In other words, water was added to the mixture, then the sample was vortexed, cooled down to room temperature and analyzed. As shown in FIG. 2B, both PEG molecular weight (200-600 Da) and the incubation temperature (25-60° C.) influence the encapsulation efficiency. The direct relationship between the molecular weight of PEG and encapsulation efficiency encouraged us to examine the formulation prepared by PEG1000 at 40 and 60° C. as well. It should be noted that PEG1000 is the highest possible molecular weight to obtain a liquid mixture at 40° C. for comparison. Higher molecular weights of PEG require higher processing temperatures. Two-way ANOVA suggests an extremely significant interaction between two variables i.e. PEG molecular weight and temperature (5.2% of the total variance, p<0.0001). The effect of both variables on encapsulation efficiency is extremely significant (p<0.0001). Tukey's multiple comparisons test implies a significantly higher encapsulation efficiency when the mixture is hydrated at 40° C. (p<0.0001) compared to both 25 and 60° C. for all the molecular weights of PEG. Hydration at 25° C. leads to a significantly higher encapsulation efficiency compared to 60° C. only for PEG600 (p<0.0001). PEG_{4kDa}-b-PLA_{2.2kDa} used in this example is expected to produce 25-50 nm PTX encapsulated micelles depending on the preparation process. PEG molecular weight and hydration temperature both significantly (p<0.0001) influence the average particle size as well (FIG. 2C). According to two-way ANOVA, the effect of PEG molecular weight is more pronounced—accounting for 68.9% of the total variance (vs. 16.6% for temperature). As seen in FIG. 2C, the average particle size increases with increasing the PEG molecular weight.

According to the obtained results, for all the examined molecular weights, liquid PEG containing PEG-b-PLA in saturated state offers the best outcome. The maximum encapsulation efficiency for PEG_{4kDa}-b-PLA_{2.2kDa} is achieved using PEG1000, with addition of water performed at 40° C. It should be noted that the saturation temperature and consequently the optimal temperature for fabrication may vary if one wishes to use higher molecular weights of copolymer and/or homo PEG. Although the optimization was performed using 5.0 wt. % PEG_{4kDa}-b-PLA_{2.2kDa} and rapid hydration (vortexing), the observed trend was found to be reproducible for other concentrations and hydration criteria. We experimented i) rapid hydration of 10.0 wt. % PEG_{4kDa}-b-PLA_{2.2kDa} with the same target loading (i.e. 33%) and ii) a slow hydration process consisting of rapid prehydration at the same temperature followed by complete hydration in a rotary mixer at 50 rpm for both 5.0 and 10.0 wt. % PEG_{4kDa}-b-PLA_{2.2kDa}. The results are displayed in FIGS. 3A-3C. The same conclusion can be made for the supplementary experiments: using higher molecular weight of PEG and hydration at 40° C. gives the highest encapsulation efficiency. Lastly, we studied the effect of reducing PTX target loading and PTX final concentration in water on the encapsulation efficiency by rapid hydration of 5.0 wt. % PEG_{4kDa}-b-PLA_{2.2kDa} in PEG600-1000 at 40° C. As illustrated in FIG. 2D, the present method with the optimal parameters can produce PTX-loaded PEG_{4kDa}-b-PLA_{2.2kDa} with almost 100% encapsulation efficiency for 10 and 20 wt. % target loading at 1 mg/mL target drug level. PEG 1000 still slightly excels PEG 600. Given the collected data, we selected to prepare the micelles at 20 wt. % target loading using 5.0 wt. % PEG-b-PLA, PEG1000 and rapid hydration at 40° C. as the optimal preparation parameters for the remainder of the experiments.

### Example 2—Mechanistic Study of Micelle Formation Reveals its Connection with Drug Encapsulation Efficiency

In order to gain a better understanding of how the present methods (also referred to as “PEG-assist”) efficiently produces drug-loaded polymeric micelles, four important questions were investigated: i) does homo PEG play a solubilization role in the system, or is drug mainly encapsulated in the micelles' core? ii) how does hydration of copolymer/PEG/drug mixture lead to formation of drug-loaded micelles? iii) how does increasing the homo PEG molecular weight improve encapsulation efficiency? iv) how does saturation temperature (e.g. 40° C.) outperform undersaturation (e.g. 60° C.) and supersaturation (e.g. 25° C.)?

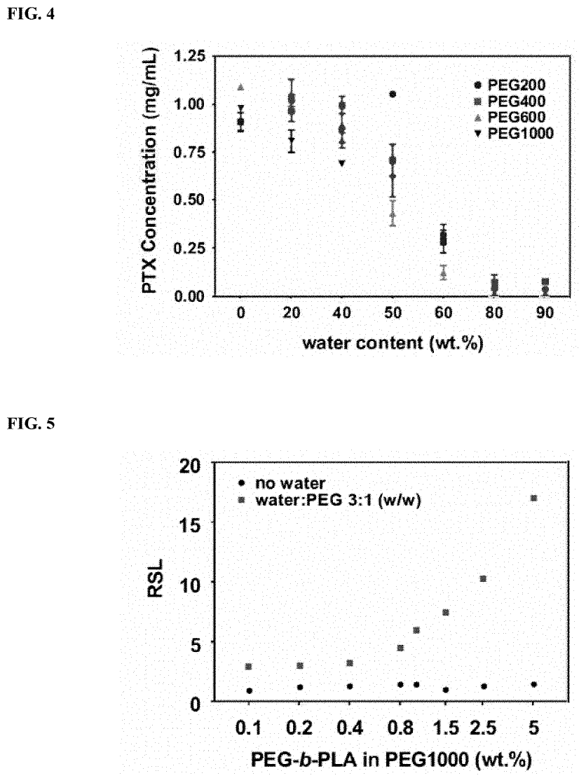

To begin with, we measured the solubility of PTX in mixtures of water and PEG of different molecular weights as a function of water content. Hydrophobic drugs such as PTX are highly soluble in liquid PEG. Addition of water, however, if surpasses a critical point, will result in precipitation of PTX (see FIG. 4). This critical point is positioned near 50.0 wt. % water content (or water:PEG 1:1 w/w ratio) for all the examined molecular weights. In other words, presence of equal or larger amount of water in a PEG-water mixture prohibits PTX solubility. Considering the PEG-assist preparation procedure, the water content always well exceeds the critical value in the final product (ca. 85.0, 92.0 or 96.0 wt. % water content at 1 mg/mL PTX level for 10, 20 or 33 wt. % target loading, respectively). Therefore, PEG-water mixture cannot play any solubilization role for PTX in the proposed system, and the drug is expected to mainly reside in the PEG-b-PLA micelles' core.

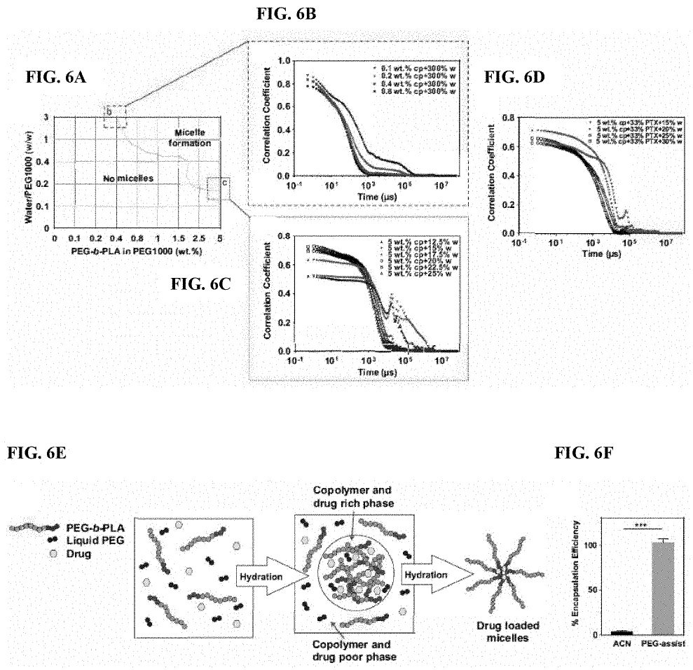

Next, we used DLS to study micelle formation near the CMCs at specific water:PEG ratios and CWCs at specific PEG-b-PLA concentrations in PEG. Those critical values can be estimated by recording the intensity of scattered light by each sample [[Topel, Ö., et al., “Determination of critical micelle concentration of polybutadiene-block-poly (ethyleneoxide) diblock copolymer by fluorescence spectroscopy and dynamic light scattering” J. Molecular Liquids, 2013. 177:40-43; Note, M. A., Surfactant Micelle Characterization using Dynamic Light Scattering. J. Phys. Chem. B, 2006:1-5; Croy, S. R. and G. S. Kwon, The effects of Pluronic block copolymers on the aggregation state of nystatin. J. Control. Release, 2004. 95(2):161-1711. To eliminate the variations caused by ratio change in our bicomponent dispersant i.e. PEG-water for different samples, we defined the parameter “RSL” which is obtained by normalizing the intensity of scattered light by sample to its dispersant. When the micelles form, a sharp increase in RSL is observed as shown in FIG. 5. The onset of RSL increase was determined for different concentrations of PEG_{4kDa}-b-PLA_{2.2kDa} in PEG1000 in various water:PEG ratios at 40° C. to generate micelle formation diagram as illustrated in FIG. 6A. In the red region of the diagram, the PEG-b-PLA and water concentrations are adequate for micelle formation. For instance, the CMC in 3:1 (w/w) water:PEG mixture is reached when the initial PEG-b-PLA concentration in PEG1000 is ca. 0.4 wt. % or higher (red dashed box in FIG. 6A). For initial concentration of 5.0 wt. % PEG-b-PLA in PEG1000, the CWC is between 10-20% water:PEG weight ratio (dashed box in FIG. 6A).

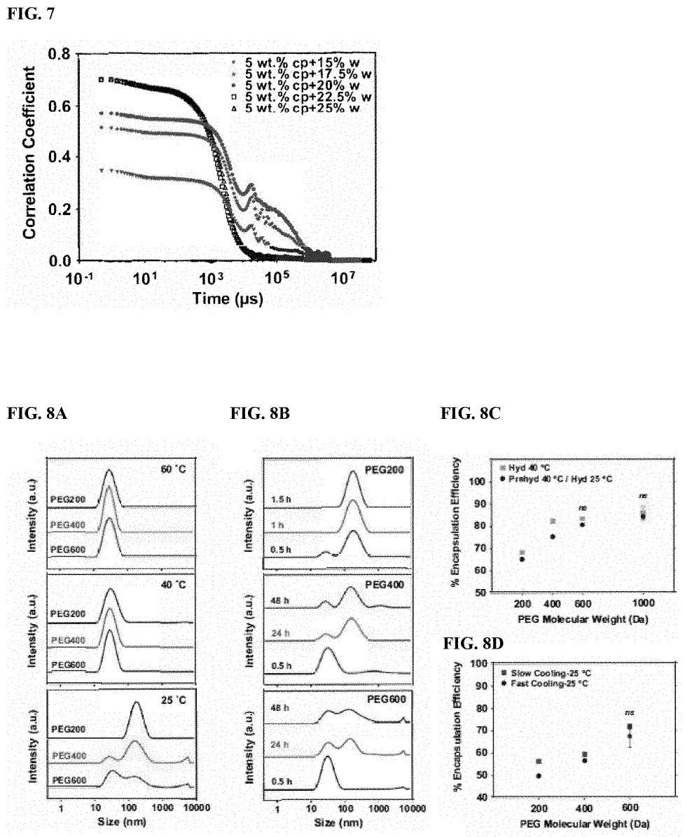

We then analyzed the correlation functions in the vicinity of critical concentrations, CMC and CWC, at 40° C. FIG. 6B displays the correlation coefficient for varied concentrations of PEG-b-PLA in 3:1 (w/w) water:PEG. The presence of large entities with long decay times is evident for low initial PEG-b-PLA concentration (0.1 wt. %). When the initial PEG-b-PLA concentration is increased to 0.2 wt. %, the signal for those entities deteriorates. The large entities vanish when the concentration is further increased to 0.4 wt. % which is the onset of RSL jump as well (FIG. 6A, dashed box). For higher concentrations, the correlation coefficient represents a uniform dispersion of PEG-b-PLA micelles with short decay times (FIG. 6B). Similarly, large entities with long decay times can be detected in 5.0 wt. % copolymer mixed with water when the water concentration is below CWC (FIG. 6C). In this case, the signal for large entities intensifies by addition of water up to 15.0% water:PEG w/w. The signal partially vanishes when the water concentration is increased to 17.5% and completely disappears for 20.0% water or higher. The onset of large entities evanescence (i.e 17.5% water) accords the onset of RSL jump (FIG. 6A, dotted box). For higher water concentrations, the correlation coefficient represents a uniform dispersion of PEG-b-PLA micelles with short decay times (FIG. 6C). Very similar trend can be observed even if a high content of PTX is added to the mixture (FIG. 6D). It should be noted that shorter decay times of PEG-b-PLA micelles in FIG. 6B compared to FIG. FIG. 6C is due to the lower viscosity of the dispersant (containing 300.0% vs. 25.0% water). The mixture behaves very similarly in the vicinity of corresponding CWC at 60° C. (see FIG. 7). Interestingly, at 60° C., the large entities to micelle transformation occurs at slightly higher water concentrations (22.5% for 60° C. vs. 17.5% for 40° C.).

It is evident that the formation of these large entities precedes PEG-b-PLA micelle formation at both temperatures. The large entities appear to be a result of liquid-liquid phase separation prior to micelle formation as suggested by Sato and Takahashi [Competition between the micellization and the liquid-liquid phase separation in amphiphilic block copolymer solutions. Polymer J., 2017. 49(2):273-277]. Liquid-liquid phase separation during amphiphilic self-assembly has been recently probed by Ianiro et al. [Liquid-liquid phase separation during amphiphilic self-assembly. Nat. Chem., 2019. 11(4):320-328] through liquid-phase electron microscopy. Their results suggest the formation of polymer-rich liquid droplets as a precursor for polymeric micelles and vesicles when the amphiphilicity is increased by changing the solvency of the copolymer blocks. The presence of large entities with long decay times prior to micelle formation and their evanescence by further hydration (FIGS. 6A-6C) is in line with such a hypothesis. Higher water content needed for such transition at 60° C. compared to 40° C. can be attributed to improved solubility of the copolymer in PEG at 60° C. which necessitates the addition of more water to induce phase separation, alter the selectivity of the environment and prompt micelle formation. In other words, lower temperatures may facilitate liquid-liquid phase separation.

Based on the collected data, we propose a model for the PEG-assist method as illustrated in FIG. 6E. Hydration of a PEG-b-PLA, PEG and drug mixture at adequately low temperatures leads to formation of a biphasic mixture consisting of a drug/copolymer-rich and a drug/copolymer-poor phase prior to the formation of drug-loaded polymeric micelles. The drug/copolymer-rich phase efficiently transforms to drug-loaded polymeric micelles and gives high encapsulation efficiencies. If the same protocol is followed but using a common organic solvent such as acetonitrile instead of PEG, the outcome is very disappointing. FIG. 6F compares the outcome of the same protocol for PEG1000 and acetonitrile used to load 20 wt. % PTX at 1 mg/mL into PEG_{4kDa}-b-PLA_{2.2kDa} micelles. As seen, acetonitrile fails to yield drug-loaded micelles when it's rapidly replaced by water. Gradual solvent replacement is needed, like in dialysis method, to achieve encapsulation by common organic solvents such as acetonitrile. In contrast, the application of PEG as the solvent can grant full encapsulation even through rapid mixing maybe due to the formation of this biphasic mixture as an intermediate phase. Offering such a route for micelle formation makes PEG-assist a very potent technique for large scale production of drug-loaded polymeric micelles as i) no organic solvent is needed for fabrication; ii) the procedure is very simple, rapid and cost-effective; iii) the method yields high encapsulation efficiencies and uniform particle size distribution; iv) micelle characteristics can be controlled by altering the biphasic mixture through temperature and composition change.

The results obtained for dependency of encapsulation efficiency on PEG molecular weight and temperature (40 vs. 60° C.) can be explained using the proposed model. Increasing PEG molecular weight decreases PTX solubility in external PEG-rich (equivalent to copolymer-poor) phase and favors its partitioning into the copolymer-rich droplets and consequently PLA made micelle core. As a result, higher PEG molecular weight is expected to give a higher encapsulation efficiency which is compatible with the trend observed in the presented data. Lowering the temperature from 60 to 40° C. is expected to have a similar effect. In other words, the PTX solubility in PEG-rich phase is lower at 40° C., favoring drug partitioning into the copolymer-rich phase and the derived micelles. Furthermore, temperature can affect the composition of the separated phases. In a mixture with upper critical solution temperature (UCST), for example, reducing temperature can further enrich the copolymer-rich phase with the copolymer and deplete the copolymer from the copolymer-poor phase. This in turn may influence drug solubility, drug distribution between phases and eventually encapsulation. While being valid for 60 vs. 40° C., the hypothesis is not applicable to 25° C. Surprisingly, further decrease in temperature not only poses no positive effect on encapsulation but also ruins it. We conducted a set of experiments to explain the cause for higher encapsulation at 40° C. compared to 25° C. which we thought is the phase transition at latter temperature.

We assumed that the unexpected reduction in encapsulation efficiency at 25° C. is linked to supersaturation and the resulted isothermal crystallization (FIG. 1C). To examine this hypothesis, we prepared a PTX-free sample by mixing PEG_{4kDa}-b-PLA_{2.2kDa} and liquid PEG at the same ratio and hydrated it after 2 h incubation at different temperatures. Particle size analysis revealed the formation of uniform polymeric micelles when hydration was performed at 60° C. or 40° C. (FIG. 8A). In contrast, hydration of the obtained waxy sample at 25° C., depending on the PEG used, led to a unimodal distribution of large particles or a bimodal distribution composed of both polymeric micelles and larger particles as illustrated in FIG. 8A. It was known from the DSC data that crystal formation at 25° C. is a time-dependent process. To confirm that crystallization in the mixture leads to formation of large particles when hydrated, we examined the effect of incubation time at 25° C. on the particle size distribution (FIG. 8B). According to the obtained results, hydration of mixture before the onset of crystallization leads to formation of polymeric micelles whereas large particles start to appear and grow in population as crystallization proceeds over time (FIG. 8B). The onset of crystallization occurs at shorter times for lower molecular weights of PEG (FIG. 1C). The mixture made with PEG200 has the shortest onset (<0.5 h) followed by PEG400 and PEG600, respectively (both >0.5 h). As seen in FIG. 8B, hydration of PEG200 mixture after 0.5 h incubation at room temperature led to a bimodal particle size distribution composed of both sub-100 nm polymeric micelles and large particles. By increasing the crystal content through prolonging incubation times, the large particles dominated the polymeric micelles in the distribution profile. Incubation for 0.5 h which is shorter than the onset of crystallization for PEG400 and 600 made mixtures, led to formation of unimodal polymeric micelles while prolonging the incubation time produced a bimodal distribution containing large particles (FIG. 8B). The collected data convinced us that crystallization in the mixture leads to formation of large particles through hydration. The cause of lower than expected encapsulation at 25° C. might be the exclusion of those large particles by centrifugation during preparation. Hydration of the mixture at higher temperatures (e.g. 40° C.) can prevent the formation of those large particles.

We next studied the effect of cooling rate and consequent isothermal crystal formation at 25° C. on the encapsulation efficiency. Sidak's multiple comparison test implies that lowering the cooling rate (60 to 25° C. at 10° C./hr vs. quenching to 25° C.) prior to incubation has no significant effect on the encapsulation efficiency (p>0.05) as shown in FIG. 8C. We also attempted to exploit the lower solubility of PTX in PEG at 25° C. (vs. 40° C.) while preventing formation of crystals at this temperature to maximize encapsulation efficiency. To do this, we performed a multi-step procedure including a prehydration step at 40° C. followed by cooling to 25° C. and lastly, complete hydration at 25° C. Although prehydration prevents the crystal formation at 25° C., the employed strategy has no significant effect on encapsulation efficiency compared to one-step hydration at 40° C.—particularly for high molecular weight PEGs (p>0.05) (FIG. 8D). We think micelle formation as a result of prehydration at 40° C. overrides the advantage of lower PTX solubility in PEG at 25° C. The formation of micelles at very low water concentration (see FIG. 6A) explains the incompetence of prehydration strategy in increasing the encapsulation efficiency (FIG. 8D). In other words, formation of drug-encapsulated micelles through prehydration at 40° C. abolishes the influence of the cooling step on encapsulation. Therefore, complete hydration at 40° C. remains the optimal protocol for micelle preparation.

### Example 3—Stable, Scalable and Freeze-Dried PTX Formulations Based on PEG-b-PLA Micelles

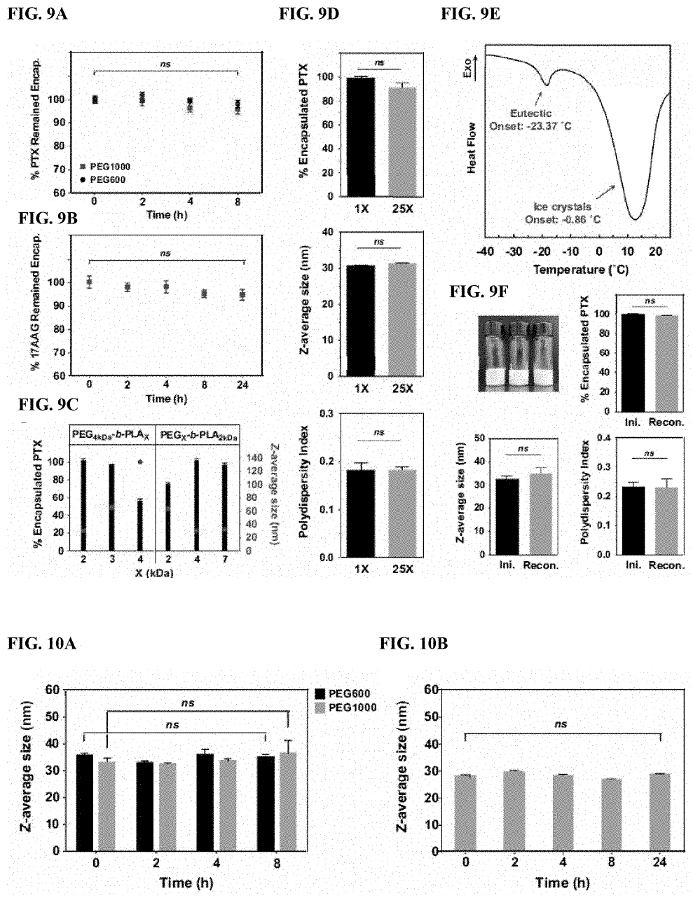

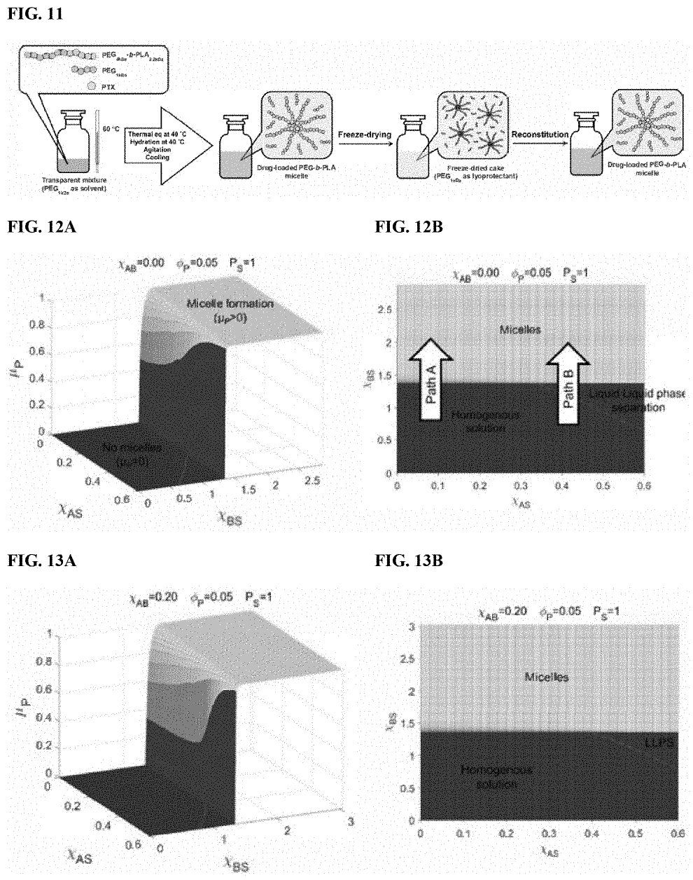

We considered stability, applicability to other drugs and copolymers, scalability and long-term preservation as four more criteria to evaluate the PEG-assist method. The stability of PTX-loaded PEG-b-PLA micelles prepared at 20 wt. % target loading with 1 mg/mL target PTX level is shown in FIG. 9A. All the samples were prepared at saturation temperature i.e. 40° C. The effect of molecular weight of the homo PEG used is also demonstrated. As seen, the formulation is stable at room temperature for at least 8 hours after hydration. Two-way ANOVA suggests that within the examined time range, PEG molecular weight and incubation time have no significant effect on the PTX content (p>0.05). In other words, PTX remains soluble after 8 hours incubation at room temperature without any significant loss for any of the PEG molecular weights (p>0.05). Particle size analysis also reveals no significant change in average size over the studied time course (see FIG. 10). We observed that all the samples were completely transparent without any visual precipitation within the 8 h time frame. It should be noted that PTX, when loaded physically, may prove unstable beyond this time scale. The inherent incompatibility of PTX with the micelle core can be alleviated using acyl and ester prodrug strategies for physical loading purposes [Tam, Y. T., et al., “Poly (ethylene glycol)-block-poly (d, 1-lactic acid) micelles containing oligo (lactic acid) 8-paclitaxel prodrug: In Vivo conversion and antitumor efficacy” J. Control. Rel., 2019. 298:186-193; Tam, Y. T., J. Gao, and G. S. Kwon, “Oligo (lactic acid) n-paclitaxel prodrugs for poly (ethylene glycol)-block-poly (lactic acid) micelles: loading, release, and backbiting conversion for anticancer activity” J. Am. Chem. Soc., 2016. 138(28):8674-8677; Repp, L., et al., “Acyl and oligo(lactic acid) prodrugs for PEG-b-PLA and PEG-b-PCL nano-assemblies for injection” J. Control. Rel., 2021. 330:1004-1015; Forrest, M. L., et al., “Paclitaxel prodrugs with sustained release and high solubility in poly (ethylene glycol)-b-poly (ε-caprolactone) micelle nanocarriers: pharmacokinetic disposition, tolerability, and cytotoxicity” Pharm. Res., 2008. 25(1):194-206; Ansell, S. M., et al., “Modulating the therapeutic activity of nanoparticle delivered paclitaxel by manipulating the hydrophobicity of prodrug conjugates” J. Med. Chem., 2008. 51(11): p. 3288-3296.]

As the proof of concept for applicability to other drugs, we used PEG-assist (PEG1000, 40° C.) to encapsulate 17AAG at a target loading and target drug level of 20 wt. % and 1 mg/mL, respectively. 17AAG can be encapsulated at 80.2±2.1% efficiency and prove stable for at least 24 h (FIG. 9B) with no visual precipitation and no significant drop in encapsulated drug content as confirmed by one-way ANOVA (p>0.05). Particle size analysis further supports the stability within the examined time frame as no significant change in average size is observed (FIG. 10).

We applied the PEG-assist method using PEG1000 at 40° C. to PEG-b-PLA block copolymers with different molecular weights of the two blocks. First, the effect of changing the molecular weight of the PLA block while keeping the PEG block molecular weight at 4 kDa was investigated. As seen in FIG. 9C, increasing the PLA block molecular weight reduces the PTX encapsulation and increases the particle size. Overall, the encapsulation efficiency remains high (>95.0%) when the PLA block molecular weight is smaller than PEG block. For equal molecular weights (i.e. PEG_{4kDa}-b-PLA_{4kDa}), the encapsulation efficiency dropped down to 56.7±1.8%. We then studied the effect of PEG block molecular weight variation while keeping the PLA block molecular weight at 2 kDa. According to our results (FIG. 6A), increasing the PEG block molecular weight from 2 to 4 or 7 kDa increases the encapsulation efficiency but reduces the particle size. It should be noted that a very probable cause for such an observation is that the parameters used are the optimal ones for PEG_{4kDa} block. Using the same parameters for PEG_{2kDa} or PEG_{7kDa} blocks may result in under- or supersaturation, respectively. Therefore, it is expected that optimizing the PEG-assist method parameters for a particular copolymer gives a higher encapsulation efficiency. Overall, obtaining encapsulation efficiencies over 95.0% for most of the formulations proves the applicability of PEG-assist to different molecular weights of the PEG-b-PLA block copolymer.

Next, we evaluated the feasibility of scaling the batch size to 25×. Again, PTX target loading and final level of 20 wt. % and 1 mg/mL was used, respectively. In other words, the batch contained 25 mg PTX, 100 mg copolymer, 1875 mg PEG1000 and was hydrated with 25 mL water. As seen in FIG. 9D, 25X scale-up using PEG1000 as the solvent and 40° C. as the processing temperature led to no significant change in encapsulation efficiency, average particle size and polydispersity (p>0.05).

Lastly, the feasibility of freeze-drying was investigated for long-term storage and reconstitution of the present PTX formulation for injection. For freeze-drying, samples were fabricated at 40° C. using PEG1000. It is well-known that PEG and water form a eutectic when mixed. We first identified the eutectic melting temperature for PEG_{4kDa}-b-PLA_{2kDa}/PEG1000/water mixture. The ratios in the mixture were adjusted as required to obtain 20 wt. % target loading and 1 mg/mL target drug level for any drug of interest. The mixture was cooled down to −80° C. at 10° C./min to freeze, followed by heating at the same rate to detect the eutectic melting onset. As seen in FIG. 9E, such a mixture produces a eutectic system with a melting onset of ca. −23.4° C.

Freeze-drying of PTX formulation with 20 wt. % loading and 1 mg/mL PTX level was performed at −35° C. shelf temperature to ensure the sample temperature remains well below the eutectic melting onset. Such a protocol yields a high-quality cake that when hydrated, produces drug-encapsulated micelles that are statistically equivalent (p>0.05) in terms of encapsulation efficiency, average particle size and polydispersity index to the initial sample before freeze-drying (FIG. 9F). According to our DSC data (not shown), PEG1000 in the freeze-died cake is crystalline with a melting onset of 35.6° C., melting peak of 38.1° C. and enthalpy of fusion equal to 103.2 J/g. It should be noted that lower molecular weights of PEG such as PEG600 are impractical for freeze-drying due to very low temperatures required for solidification which in turn prohibits adequate drying on account of low ice vapor pressure at such temperatures. Practically, PEG1000 is the lowest molecular weight that can be used to freeze-dry the samples.

The optimal PEG-assist procedure to obtain a freeze-dried cake that can produce drug-loaded PEG_{4kDa}-b-PLA_{2kDa} micelles containing PTX upon reconstitution has been schematically illustrated in FIG. 11. One of the important advantages of using high molecular weight PEGs at elevated temperatures in PEG-assist over using low molecular weight liquid PEGs is the abolition of the need to remove PEG before freeze-drying. Furthermore, no other lyoprotectant is needed for successful freeze-drying. In other words, high molecular weight PEG can play a dual role, both as the solvent and the lyoprotectant, in PEG-assist method. PEG-assist can be optimized for different molecular weights of homo PEG and PEG-b-PLA block copolymer. For a different combination, hydration should be performed near the solution saturation temperature and freeze-drying done at a temperature below the eutectic point.

### Example 4—Modeling of Micelle Formation Between a Mixture of Block Copolymer A-B and Oligomer A

A free energy equation for a mixture of a block copolymer A-B and a homopolymer/oligomer A to study the micelle formation by changing the Flory Huggins interaction parameter. Both temperature and selectivity of the medium for one of the blocks of the copolymer can influence the interaction parameter and consequently micelle formation. The free energy in the present model has 3 components:
free energy of micelles (ΔGₘ, due to assembly)
free energy of solution (ΔGₕ, unimers not assembled into a micellar structure)
translational entropy of the micelles (Sₘₜ)
   Therefore, the total free energy of the system can be written as

$$\frac{Δ G}{{k}_{B} T} = \frac{Δ {G}_{m}}{{k}_{B} T} + \frac{Δ {G}_{h}}{{k}_{B} T} - \frac{{TS}_{mt}}{{k}_{B} T}$$

Based on prior work [Sato, T. and R. Takahashi, Competition between the micellization and the liquid-liquid phase separation in amphiphilic block copolymer solutions. Polymer Journal, 2017. 49(2): p. 273-277; Leibler, L., H. Orland, and J. C. Wheeler, Theory of critical micelle concentration for solutions of block copolymers. J. Chem. Physics, 1983. 79(7): p. 3550-3557], a comprehensive model for micelle formation was developed for a mixture of block copolymer A-B and homopolymer or oligomer A. The free energy of the system can be written as:

$$\frac{Δ g}{{k}_{B} T} = \frac{{μ}_{P} {ϕ}_{P}}{{ϕ}_{Pm}} [ \text{} \frac{{ϕ}_{Pm}}{{P}_{A} + {P}_{B}} ( ln κ + ln {ϕ}_{Pm} ) + \frac{{x}_{A}}{{P}_{S} {τ}_{1}} ( 1 - {τ}_{1} {ϕ}_{Pm} ) ln ( 1 - {τ}_{1} {ϕ}_{Pm} ) + \frac{{x}_{B}}{{P}_{S} {τ}_{2}} ( 1 - {τ}_{2} {ϕ}_{Pm} ) ln ( 1 - {τ}_{2} {ϕ}_{Pm} ) + [ \text{} {x}_{A} ( 1 - {τ}_{1} {ϕ}_{Pm} ) {χ}_{AS} + {x}_{B} ( 1 - {τ}_{2} {ϕ}_{Pm} ) {χ}_{BS} - {x}_{A} {x}_{B} {χ}_{AB} ] {ϕ}_{Pm} + \frac{3 {x}_{B}}{{τ}_{2} {P}_{B}^{α}} Γ ] + ( 1 - \frac{{μ}_{P} {ϕ}_{P}}{{ϕ}_{Pm}} ) [ \text{} \frac{1 - {ϕ}_{Ph}}{{P}_{S}} ln ( 1 - {ϕ}_{Ph} ) + \frac{{ϕ}_{Ph}}{{P}_{A} + {P}_{B}} ln {ϕ}_{Ph} + \overline{χ} ( 1 - {ϕ}_{Ph} ) {ϕ}_{Ph} ] + [ \text{} \frac{{μ}_{P} {ϕ}_{P}}{{N}_{P} ( {P}_{A} + {P}_{B} )} ln ( \frac{{μ}_{P} {ϕ}_{P}}{{ϕ}_{Pm}} ) + \frac{{ϕ}_{Pm} - {μ}_{P} {ϕ}_{P}}{{N}_{P} ( {P}_{A} + {P}_{B} )} ln ( 1 - \frac{{μ}_{P} {ϕ}_{P}}{{ϕ}_{Pm}} ) ]$$

where,
k_{B} is the Boltzmann constant;
T is the absolute temperature;
A is the solvophilic block of diblock copolymer (P);
B is the solvophobic block of diblock copolymer (P);
P_{A} and P_{B} are the degree of polymerization of block A and block B of the diblock copolymer (A−B), respectively; the degree of polymerization for the copolymer would be P=P_{A}+P_{B}; P=P_{A}+P_{B}
P_{S} is the degree of polymerization of the homopolymer (S);
ϕₚ is the volume fraction of the diblock copolymer in the entire system;
ϕₚₕ is the volume fraction of the diblock copolymer in the mixture;
α is the correction factor for the projection length of the blocks;
χ̅ is the average interaction parameter between the diblock copolymer and the homopolymer and is calculated as:

χ̅≡x_{A}χ_{AS}−x_{B}χ_{BS}−x_{A}x_{B}χ_{AB}

where,
X_{AS}, X_{BS} and X_{AB} are the interaction parameters between the doublets of A, B and S;
x_{A} and x_{B} are the mole fractions of A and B monomers in the diblock copolymer and are calculated as:

$${x}_{A} = 1 - {x}_{B} = \frac{{P}_{A}}{{P}_{A} + {P}_{B}}$$

μp is the fraction of copolymer chains aggregated in spherical micelles;
ϕ_{Pm} is the volume fraction of copolymer chains in a single micelle;

$$κ = \frac{π}{27} {P}_{A} {{P}_{B}^{1 - 2 α} ( {P}_{A}^{α} + {P}_{B}^{α} )}^{3}$$

$${τ}_{1} = \frac{{( {x}_{B}^{α} + {x}_{A}^{α} )}^{3} {x}_{A}}{{( {x}_{B}^{α} + {x}_{A}^{α} )}^{3} - {x}_{B}^{3 α}} and {τ}_{2} = \frac{{( {x}_{B}^{α} + {x}_{A}^{α} )}^{3}}{{x}_{B}^{3 α - 1}}$$

$$Γ = \sqrt{\frac{1}{3} ( {τ}_{1} {ϕ}_{Pm} + {τ}_{2} {ϕ}_{Pm} ) \{ \begin{matrix}[ ln ( 1 - \frac{{τ}_{1}}{2} {ϕ}_{Pm} - \frac{{τ}_{2}}{2} {ϕ}_{Pm} ) + \frac{{τ}_{1}}{2} {ϕ}_{Pm} {χ}_{AS} + \\ \frac{{τ}_{2}}{2} {ϕ}_{Pm} {χ}_{BS} ] ( 1 - \frac{{τ}_{1}}{2} {ϕ}_{Pm} - \frac{{τ}_{2}}{2} {ϕ}_{Pm} ) + \frac{{τ}_{1} {τ}_{2}}{4} {ϕ}_{Pm}^{2} {χ}_{AB} - \\ \frac{1}{2} [ \begin{matrix}[ ln ( 1 - {τ}_{1} {ϕ}_{Pm} ) + {τ}_{1} {ϕ}_{Pm} {χ}_{AS} ] ( 1 - {τ}_{1} {ϕ}_{Pm} ) + \\ [ ln ( 1 - {τ}_{2} {ϕ}_{Pm} ) + {τ}_{2} {ϕ}_{Pm} {χ}_{BS} ] ( 1 - {τ}_{2} {ϕ}_{Pm} )\end{matrix} ]\end{matrix} \}}$$

$${ϕ}_{Ph} = \frac{( 1 - {μ}_{P} ) {ϕ}_{P}}{( 1 - \frac{{μ}_{P} {ϕ}_{P}}{{ϕ}_{Pm}} )}$$

$${N}_{P} = \frac{4 π {{ϕ}_{Pm} ( {P}_{A}^{α} + {P}_{B}^{α} )}^{3}}{3 ( {P}_{A} + {P}_{B} )}$$

Therefore, Δg/k_{B}T in such a system depends on 4 variables i.e. μₚ, ϕₚ, ϕ_{Pm}, and χ̅ and the thermodynamically stable states of the system can be predicted by minimizing the free energy per lattice site with respect to those 4 variables. The free energy equation was solved/minimized with respect to μₚ and ϕ_{Pm} φ_{Pm} for different values of ϕₚ and χ̅ using matlab. When both pp and ϕ_{Pm} get non-zero values (μₚ>0 and ϕ_{Pm}>0), it means that micelles have formed. Otherwise when (μₚ>0 and ϕₚₘ>0), no micelles exist and one has either a homogeneous solution or liquid liquid phase separation (LLPS). When there are no micelles, the system undergoes LLPS if the value for χ̅ exceeds a critical value of average interaction parameter (χ̅_{c}) that can be calculated as:

$${\overline{χ}}_{c} ≡ {\frac{1}{2} [ \frac{1}{\sqrt{{P}_{A} + {P}_{B}}} + \frac{1}{\sqrt{{P}_{S}}} ]}^{2}$$

FIGS. 12A and 12B show sample data for a block copolymer (P_{A}=90, P_{B}=30) with X_{AB}=0 and a small molecule solvent (P_{S}=1) when block copolymer A-B is mixed with the solvent at 5 wt. % concentration. FIG. 12B shows the top view.

Starting from a homogenous solution (which is obtained by dissolving copolymer A-B in solvent S), depending on the values of interaction parameter X_{AS} which is dictated by the copolymer nature and the solvent nature and interaction between them, micelles may be obtained directly from homogenous solution or after the system undergoes liquid liquid phase separation. By changing the copolymer type (X_{AB}=0.2) but still using a small molecule solvent, no significant change in LLPS region is observed and still micelles may be obtained directly from homogenous solution depending on the path followed (see FIGS. 13A, 13B).

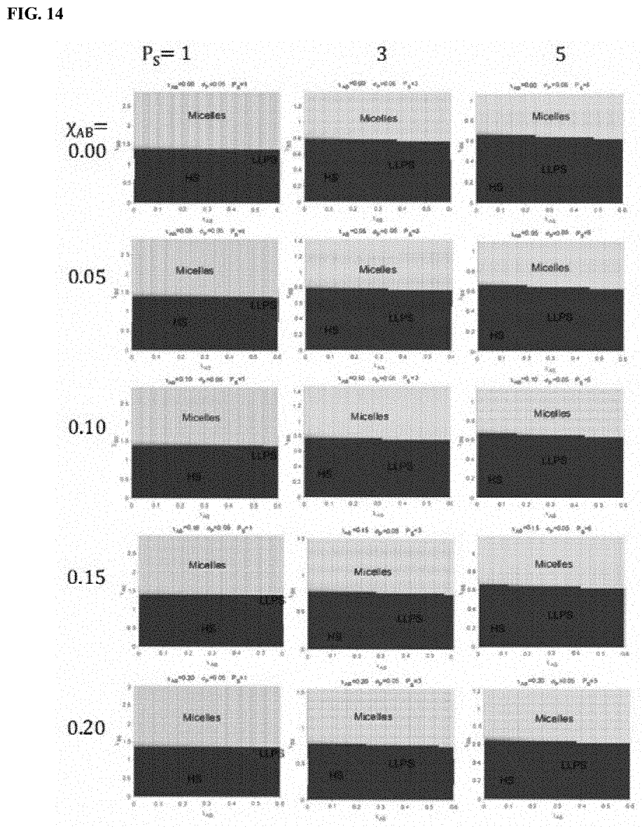

By increasing the degree of polymerization of the solvent i.e. using a homopolymer or oligomer as the solvent, the LLPS region can be significantly manipulated. FIG. 14 displays the outcome of modeling for different χ_{AB} values ranging from 0-0.2 and different degrees of polymerization for solvent (P_{S}=1 to 5).

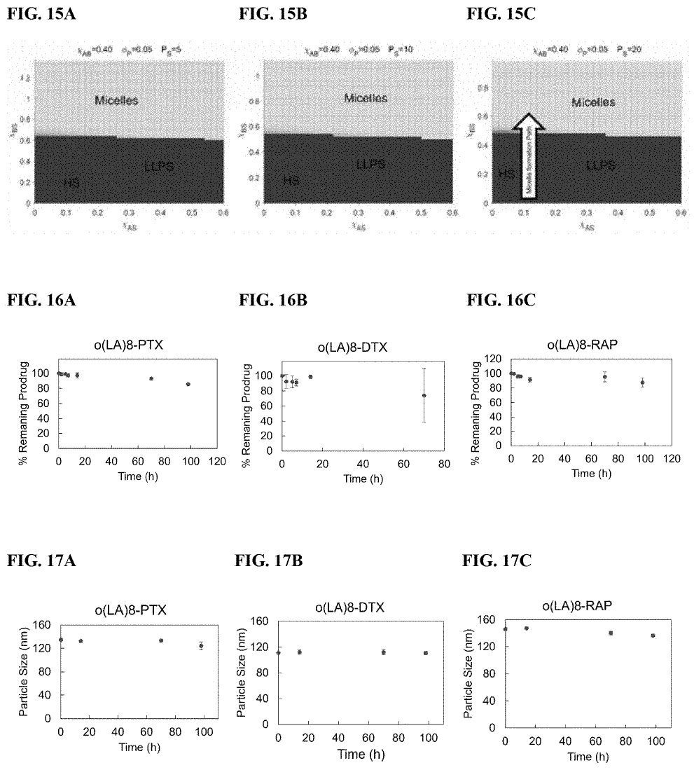

As seen, the LLPS region can be expanded for any X_{AB} by increasing the degree of polymerization for solvent (P_{S}) to an extent that LLPS always precedes micelle formation and obtaining micelles from homogenous solution becomes impossible. Even for high X_{AB} values, such as X_{AB}=0.4, there is a critical value for P_{S} P_{S} above which LLPS is unavoidable. FIGS. 15A-C shows how high X_{AB} requires the application of higher degree of polymerization (P_{S}>20) of homopolymer/oligomer as the solvent. Table 2 shows the estimated values of X_{AB} χ_{AB} for different copolymers according to Iarino et al. [“Liquid-liquid phase separation during amphiphilic self-assembly” Nat. Chem., 2019. 11(4):320-328].

TABLE 2

| Copolymer | χ_{AB} |
|---|---|
| Poly(ethylene oxide)-block-Poly(lactic-co-glycolic acid) | 0.06 |
| PEO-PLGA (50:50) | |
| Poly(ethylene oxide)-block-Poly(propylene oxide) | 0.09 |
| PEO-PPO | |
| Poly(ethylene oxide)-block-Polystyrene PEO-PS | 0.43 |
| Poly(ethylene oxide)-block-Poly(methyl methacrylate) | 0.1 |
| PEO-PMMA | |
| Poly(ethylene oxide)-block-Poly-L-lactide PEO-PLLA | 0.02 |
| Poly(vinyl alcohol)-block-Poly-ε-caprolactone PVOH-PCL | 0.29 |
| Poly(vinyl alcohol)-block-Poly(lactic-co-glycolic acid) | 0.08 |
| PVOH-PLGA (50:50) | |
| Poly(vinyl alcohol)-block-Poly(propylene oxide) | 0.15 |
| PVOH-PPO | |
| Poly(vinyl alcohol)-block-Polystyrene PVOH-PS | 0.38 |
| Poly(vinyl alcohol)-block-(methyl methacrylate) | 0.17 |
| PVOH-PMMA | |
| Poly(vinyl alcohol)-block-Polypropylene PVOH-PLLA | 0.11 |

Therefore, a copolymer with any X_{AB} can be mixed with/dissolved in a homopolymer/oligomer A with a certain degree of polymerization, undergo liquid liquid phase separation independent of χ_{AS} X_{AB} value, and produce micelles by addition of water. The mixture can be made of copolymer A-B and homopolymer/oligomer A or copolymer A-B mixed with homopolymer/oligomer C. As shown herein, PEG is an example of a homopolymer/oligomer that can be used to provide micelles. Other oligomers and copolymers also follow very similar rules since the model is applicable regardless of the copolymer/oligomer pair. The values of X_{AB}, X_{AB} and X_{BS} indicate the nature of copolymer/oligomer and interaction between them.

### Liquid Liquid Phase Separation

Under certain circumstances, depending on temperature and interaction parameter between components, a polymer solution or a polymer blend may undergo liquid liquid phase separation (LLPS). It means that for certain concentrations of polymer, a homogeneous solution is not the most thermodynamically stable state of the system. In this case, the system becomes metastable or unstable. In metastable state, occurs in so-called binodal region, two phases, one polymer rich and one polymer poor, form by means of nucleation and growth mechanism. In such regime, droplets of one phase considered as the disperse phase start to form and grow in the other phase considered as continuous phase. If the polymer concentration falls within the range known as the unstable region, the solution undergoes spinodal decomposition. In this case polymer rich and polymer poor phases form as two co-continuous phases [Deneau, E. and G. Steele, “An in-line study of oiling out and crystallization” Organic process research & development, 2005. 9(6):943-950; Hiemenz, P. C. and T. P. Lodge, Polymer Chemistry 2007: CRC press.]. According to our model, the present formulations comprising a block copolymer and a homopolymer/oligomer as the solvent undergo LLPS prior to micelle formation.

### Crystallization

PEG is a crystallizable polymer. So the PEG block of a copolymer can crystallize if cooled. If the copolymer is mixed with a homopolymer/oligomer, crystallization can occur from solution. In this case, when the solution is cooled down (from 60° C. for example), at a certain temperature (which is below 40° C. for the examples in our invention disclosure), PEG block of the copolymer crystallizes out of the solution. This leads to formation of a biphasic mixture, one the crystallized copolymer and the other an undersaturated solution of the copolymer. hydration of this biphasic mixture may lead to formation of non-uniform particles with wide size distribution. Exclusion of large particles during filter sterilization or centrifugation may lower encapsulation efficiency. Furthermore, for a mixture of copolymer/oligomer, the system may undergo LLPS with cooling. Therefore, the system may experience LLPS prior to crystallization. High supersaturation in this case may make the crystallization difficult to control, polymorphism and production of crystals with different shape and sizes which in turn impact the homogeneity of the final product.

Therefore, crystallization is avoided by addition of water at a temperature higher that crystallization temperature of the copolymer (determined by DSC). In our examples, the ideal hydration temperature is 40° C. but this temperature may need to change depending on the type and molecular weight of the copolymer used.

### Eutectic

Sample needs to remain solid during primary drying in lyophilization process. When freezing a sample for lyophilization, depending on the composition, a glassy matrix or a semi-crystalline/crystalline matrix may form. Amorphous systems form a glassy matrix with T′g (glass transition temperature) while crystallizable ones form a crystalline/semi-crystalline matrix with Tₑ known as eutectic temperature. T′_{g} and Tₑ represent the highest temperature that is safe for the product during primary drying [Amin, K., et al., Lyophilization of polyethylene glycol mixtures. Journal of pharmaceutical sciences, 2004. 93(9): p. 2244-2249].

PEG in water forms a eutectic mixture [Kuttich, B., et al., X-ray scattering study on the crystalline and semi-crystalline structure of water/PEG mixtures in their eutectic phase diagram. Soft Matter, 2020. 16(45): p. 10260-10267]. When aqueous solution of PEG is cooled down, first pure ice crystals form and PEG gets concentrated in the solution. In this case, two phases co-exist; pure ice crystals and freeze-concentrated PEG solution. Further cooling below Tₑ leads to solidification of the freeze-concentrated PEG solution as well. The sample must be kept below this temperature throughout lyophilization in order to obtain a clean cake. The eutectic temperature may change by addition of additives or changing the molecular weight of PEG. Certain additives may prevent crystallization and lead to formation of a glassy matrix.

### Example 5—PEG-Assist Capability for Encapsulation of Prodrug (PTX, DTX, or RAP) Using PEG1000

### Example 5a: PEG-Assist Capability for Encapsulation of PTX Prodrug

a) 1.8 mg oLA8-PTX and 1.8 mg PEG4k-b-PLA2.2k were mixed with 36 mg PEG1000. The mixture was heated to 60° C. and kept for 30 min at this temperature. The sample was then cooled down to 40° C. and incubated for 2 h at this temperature. The sample was hydrated using 1.8 mL water at 40° C., cooled down to room temperature, centrifuged at 10000 g for 10 min and analyzed.

b) 2.7 mg oLA8-PTX and 2.7 mg PEG4k-b-PLA2.2k were mixed with 54 mg PEG1000. The mixture was heated to 60° C. and kept for 30 min at this temperature. The sample was then cooled down to 40° C. and incubated for 2 h at this temperature. The sample was hydrated using 2.7 mL water at 40° C., cooled down to room temperature, centrifuged at 10000 g for 10 min and analyzed.

c) 2.4 mg oLA8-PTX and 2.4 mg PEG4k-b-PLA2.2k were mixed with 48 mg PEG1000. The mixture was heated to 60° C. and kept for 30 min at this temperature. The sample was then cooled down to 40° C. and incubated for 2 h at this temperature. The sample was hydrated using 2.4 mL water at 40° C., cooled down to room temperature, centrifuged at 10000 g for 10 min and analyzed.

The measurement for each sample was repeated twice. Table 3 shows the results and average encapsulation efficiency and drug loading obtained for oLA8-PTX.

TABLE 3
oLA8-PTX encapsulated in PEG4k-b-PLA2.2k using PEG1000

| Sample | % EE (Encapsulation efficiency) | % DL (Drug loading) |
|---|---|---|
| 1-a | 85.4 | 46.1 |
| | 85.7 | 46.1 |
| 1-b | 100.7 | 50.2 |
| | 101.1 | 50.3 |
| 1-c | 89.4 | 47.2 |
| | 89.7 | 47.3 |
| Average | 92.0 ± 7.1 | 47.9 ± 1.9 |

### Example 5b: PEG-Assist Capability for Encapsulation of DTX Prodrug

a) 1.6 mg oLA8-DTX and 1.6 mg PEG4k-b-PLA2.2k were mixed with 32 mg PEG1000. The mixture was heated to 60° C. and kept for 30 min at this temperature. The sample was then cooled down to 40° C. and incubated for 2 h at this temperature. The sample was hydrated using 1.6 mL water at 40° C., cooled down to room temperature, centrifuged at 10000 g for 10 min and analyzed.

b) 1.9 mg oLA8-DTX and 1.9 mg PEG4k-b-PLA2.2k were mixed with 38 mg PEG1000. The mixture was heated to 60° C. and kept for 30 min at this temperature. The sample was then cooled down to 40° C. and incubated for 2 h at this temperature. The sample was hydrated using 1.9 mL water at 40° C., cooled down to room temperature, centrifuged at 10000 g for 10 min and analyzed.

c) 1.4 mg oLA8-DTX and 1.4 mg PEG4k-b-PLA2.2k were mixed with 28 mg PEG1000. The mixture was heated to 60° C. and kept for 30 min at this temperature. The sample was then cooled down to 40° C. and incubated for 2 h at this temperature. The sample was hydrated using 1.4 mL water at 40° C., cooled down to room temperature, centrifuged at 10000 g for 10 min and analyzed.

The measurement for each sample was repeated twice. Table 4 shows the results and average encapsulation efficiency and drug loading obtained for oLA8-DTX.

TABLE 4
oLA8-DTX encapsulated in PEG4k-b-PLA2.2k using PEG1000

| Sample | % EE (Encapsulation efficiency) | % DL (Drug loading) |
|---|---|---|
| 2-a | 93.4 | 48.3 |
| | 92.3 | 48 |
| 2-b | 91.2 | 47.7 |
| | 92 | 47.9 |
| 2-c | 87.7 | 46.7 |
| | 87.9 | 46.8 |
| Average | 90.8 ± 2.4 | 47.6 ± 0.7 |

### Example 5c: PEG-Assist Capability for Encapsulation of RAP Prodrug

a) 1.6 mg oLA8-RAP and 1.6 mg PEG4k-b-PLA2.2k were mixed with 32 mg PEG1000. The target loading was 50 wt. %. The mixture was heated to 60° C. and kept for 30 min at this temperature. The sample was then cooled down to 40° C. and incubated for 2 h at this temperature. The sample was hydrated using 1.6 mL water at 40° C., cooled down to room temperature, centrifuged at 10000 g for 10 min and analyzed.

b) 1.6 mg oLA8-RAP and 1.6 mg PEG4k-b-PLA2.2k were mixed with 32 mg PEG1000. The mixture was heated to 60° C. and kept for 30 min at this temperature. The sample was then cooled down to 40° C. and incubated for 2 h at this temperature. The sample was hydrated using 1.6 mL water at 40° C., cooled down to room temperature, centrifuged at 10000 g for 10 min and analyzed.

c) 1.6 mg oLA8-RAP and 1.6 mg PEG4k-b-PLA2.2k were mixed with 32 mg PEG1000. The mixture was heated to 60° C. and kept for 30 min at this temperature. The sample was then cooled down to 40° C. and incubated for 2 h at this temperature. The sample was hydrated using 1.6 mL water at 40° C., cooled down to room temperature, centrifuged at 10000 g for 10 min and analyzed.

The measurement for each sample was repeated twice. Table 5 shows the results and average encapsulation efficiency and drug loading obtained for oLA8-RAP.

TABLE 5
oLA8-RAP encapsulated in PEG4k-b-PLA2.2k using PEG1000

| Sample | % EE (Encapsulation efficiency) | % DL (Drug loading) |
|---|---|---|
| 3-a | 82.6 | 45.2 |
| | 82.6 | 45.2 |
| 3-b | 81.2 | 44.8 |
| | 81.2 | 44.8 |
| 3-c | 89.6 | 47.3 |
| | 81.5 | 44.9 |
| Average | 83.1 ± 3.2 | 45.4 ± 1.0 |

### Example 6—Stability of Prodrug Formulations Made Using PEG1000

The samples in Example 5 were tested for stability. The samples were found to be very stable at room temperature. FIGS. 16A-16C show the percentage of prodrugs remained encapsulated over a period of 3-4 days. As seen, all the formulations retained major part of the encapsulated prodrug after 72 h at room temperature. Amongst all, o(LA)8-DTX showed the lowest stability.

We also measured particle size variations over time for the 3 formulations (FIGS. 17A-17C). As expected, no significant change in particle size was observed in 3-4 days.

The samples in Example 5 were also freeze-dried. The samples were transferred to −80° C. and kept overnight followed by lyophilization at −35° C. and 15 Pa in a VirTis Advantage Pro shelf freeze-dryer for 72 h. The samples were reconstituted using the original amount of water they contained before lyophilization and analyzed for drug loss. Table 6 shows drug loading content and encapsulation efficiency before and after lyophilization.

TABLE 6
Drug loading content and encapsulation efficiency before
and after lyophilization for oLA8-PTX, oLA8-DTX or oLA8-
RAP encapsulated in PEG-b-PLA micelles using PEG1000-assist

| | Target | Actual loading (wt. %) | | % Encapsulation efficiency | |
|---|---|---|---|---|---|
| | loading | Before | After | Before | After |
| Prodrug | (wt. %) | lyophilization | lyophilization | lyophilization | lyophilization |
| oLA8-PTX | 50 | 47.9 ± 1.9 | 33.5 ± 1.9 | 92.0 ± 7.1 | 50.5 ± 4.4 |
| oLA8-DTX | 50 | 47.6 ± 0.7 | 37.7 ± 2.1 | 90.8 ± 2.4 | 60.7 ± 5.3 |
| oLA8-RAP | 50 | 45.4 ± 1.0 | 29.4 ± 4.6 | 83.1 ± 3.2 | 42.2 ± 9.1 |

### Example 7—PEG-Assist Capability for Encapsulation Prodrug (PTX or RAP) Using Higher Molecular Weight PEG (PEG2000)

### Example 7a: PEG-Assist Capability for Encapsulation of PTX Prodrug

Example 5a was repeated using PEG2000 to encapsulate oLA8-PTX in PEG4k-b-PLA2.2k at a target loading of 50 wt. %. Briefly, ca. 1 mg prodrug and 1 mg copolymer were mixed with 20 mg PEG2000, heated to 60° C. and hydrated at the same temperature after 2 h using ca. 1 mL water. Samples were cooled down to room temperature, centrifuged at 10000 g for 10 min and analyzed (n=3, mean±SD). Encapsulation efficiency, drug loading and particle size were 95.4±6.2% w/w, 47.7±3.1 wt. % and 134.7±3.3 nm, respectively.

### Example 7b: PEG-Assist Capability for Encapsulation of RAP Prodrug

Example 5c was repeated using PEG2000 to encapsulate oLA8-RAP in PEG4k-b-PLA2.2k at a target loading of 50 wt. %. Briefly, ca. 1 mg prodrug and 1 mg copolymer were mixed with 20 mg PEG2000, heated to 60° C. and hydrated at the same temperature after 2 h using ca. 1 mL water. Samples were cooled down to room temperature, centrifuged at 10000 g for 10 min and analyzed (n=3, mean±SD). Encapsulation efficiency, drug loading and particle size were 94.4±3.7% w/w, 47.2±1.8 wt. % and 146.1±2.5 nm, respectively.

### Example 8—PEG-Assist Capability for Encapsulation of Multiple Prodrugs in a Single Micelle Using Higher Molecular Weight PEG (PEG2000)

Multiple-prodrug-loaded micelles loaded with oLA8-PTX and oLA8-RAP were prepared using the same parameters as Example 7 (n=3, mean±SD). In this case, the target prodrug loading was 50 wt. % which at a 5:1 molar ratio is equivalent to ca. 42% o(LA)₈-PTX and ca. 8% o(LA)₈-RAP. The encapsulation efficiencies for o(LA)₈-PTX and o(LA)₈-RAP when co-loaded in PEG4k-b-PLA2.2k using PEG2000 were 98.6±3.3% and 97.7±4.4%, respectively. The total prodrug weight loading was 49.2±1.7 wt. % consisting of 41.1±1.4 o(LA)₈-PTX and 8.2±0.4 o(LA)₈-RAP. The particle size for multiple-prodrug-loaded micelles was 109.1±5.6 nm. Table 7 shows the results for Examples 7-8.

TABLE 7
Drug loading content, encapsulation efficiency and particle size for oLA8-
PTX, oLA8-RAP or co-encapsulated oLA8-PTX/oLA8-RAP using PEG2000-assist

| | % Encapsulation | | | | |
|---|---|---|---|---|---|
| | efficiency | | % Drug loading | | Particle size |
| Sample | oLA8-PTX | oLA8-RAP | oLA8-PTX | oLA8-RAP | (nm) |
| Individual oLA8-PTX | 95.4 ± 6.2 | — | 47.7 ± 3.1 | — | 134.7 ± 3.3 |
| Individual oLA8-RAP | — | 94.4 ± 3.7 | — | 47.2 ± 1.8 | 146.1 ± 2.5 |
| Co-loaded | 98.6 ± 3.3 | 97.7 ± 4.4 | 41.1 ± 1.4 | 8.2 ± 0.4 | 109.1 ± 5.6 |
| oLA8-PTX/oLA8-RAP | | | | | |

### Example 9—PEG-Assist Capability for Encapsulation of Multiple Prodrugs with Different Ratios in a Single Micelle

Using the same fabrication parameters but different ratios, oLA8-PTX and oLA8-RAP were loaded into PEG4k-b-PLA2.2k using PEG2000. Table 8 shows the results for encapsulation efficiency, drug loading and particle size.

TABLE 8
Drug loading content, encapsulation efficiency and particle
size for oLA8-PTX, oLA8-RAP or co-encapsulated oLA8-PTX/oLA8-
RAP at different ratios using PEG-assist method

| oLA₈- | | | | | Total |
|---|---|---|---|---|---|
| PTX/oLA₈- | | Encapsulation | | Loaded | weight |
| RAP molar | Size (nm) | efficiency | Weight loading | prodrug | loading |
| ratio | (PDI) | (% w/w) | (wt. %) | mole ratio | (wt. %) |
| 6:0 | 105.9 ± 42.9 | oLA₈-PTX: | oLA₈-PTX: | — | 48.4 ± 4.4 |
| | (0.245) | 93.7 ± 4.6 | 48.4 ± 4.4 | | |
| | | oLA₈-RAP: 0 | oLA₈-RAP: 0 | | |
| 5:1 | 104.7 ± 43.3 | oLA₈-PTX: | oLA₈-PTX: | 5.2:1 | 48.4 ± 3.1 |
| | (0.275) | 94.3 ± 3.6 | 40.4 ± 2.9 | | |
| | | oLA₈-RAP: | oLA₈-RAP: | | |
| | | 95.5 ± 1.2 | 8.2 ± 0.2 | | |
| 4:2 | 112.4 ± 58.0 | oLA₈-PTX: | oLA₈-PTX: | 2.1:1 | 49.6 ± 3.0 |
| | (0.224) | 98.2 ± 2.8 | 33.0 ± 1.8 | | |
| | | oLA₈-RAP: | oLA₈-RAP: | | |
| | | 98.9 ± 3.8 | 16.6 ± 1.2 | | |
| 3:3 | 118.9 ± 41.7 | oLA₈-PTX: | oLA₈-PTX: | 1.1:1 | 49.1 ± 4.0 |
| | (0.243) | 98.1 ± 3.8 | 24.9 ± 1.8 | | |
| | | oLA₈-RAP: | oLA₈-RAP: | | |
| | | 95.2 ± 4.6 | 24.2 ± 2.2 | | |
| 2:4 | 106.7 ± 37.6 | oLA₈-PTX: | oLA₈-PTX: | 1:1.9 | 49.1 ± 4.8 |
| | (0.191) | 96.0 ± 5.5 | 16.3 ± 1.7 | | |
| | | oLA₈-RAP: | oLA₈-RAP: | | |
| | | 96.8 ± 4.8 | 32.8 ± 3.0 | | |
| 1:5 | 107.3 ± 41.3 | oLA₈-PTX: | oLA₈-PTX: | 1:4.9 | 48.3 ± 3.1 |
| | (0.136) | 92.1 ± 3.9 | 7.9 ± 0.6 | | |
| | | oLA₈-RAP: | oLA₈-RAP: | | |
| | | 93.6 ± 3.1 | 40.3 ± 2.5 | | |
| 0:6 | 98.2 ± 32.9 | oLA₈-PTX: 0 | oLA₈-PTX: — | — | 49.5 ± 4.9 |
| | (0.126) | oLA₈-RAP: | oLA₈-RAP: | | |
| | | 97.9 ± 5.1 | 49.5 ± 4.9 | | |

### Example 10—In Vivo Anticancer Activity of the Formulations Prepared by PEG-Assist Method

Balb/c mice (female, 6 and 8 weeks old) were purchased from Jackson Lab. The mice were housed with free access to food and water in the animal room and were kept at a constant temperature (23±1° C.) and relative humidity (60±10%) for 1 week. 4T1 cells (1×10⁶ cells, suspended in 100 μL serum-free DMEM) were subcutaneously injected behind each mouse's flank.

Two weeks following tumor inoculation, mice were randomly divided into 6 groups (n=5 per group) and intravenously administered (i) saline control, (ii) o(LA)8-PTX-loaded micelles, (iii) o(LA)8-RAP-loaded micelles, (iv) PTX:RAP 5:1 co-loaded drug micelles, (v) Abraxane as commercial benchmark, and (vi) o(LA)8-PTX:o(LA)8-RAP 5:1 co-loaded prodrug micelles. Mice were injected weekly for 3 weeks followed by 1 week of observation. Mice were dosed at 100 μL saline, 50 mg/kg oLA8-PTX, 11 mg/kg oLA8-RAP, 50 mg/kg and 11 mg/kg PTX/RAP, 50 mg/kg Abraxane, 50 mg/kg and 11 mg/kg oLA8-PTX/oLA8-RAP. Body weights and tumor volumes were monitored throughout the study every 2-3 days.

The blood was collected in Retro-orbital after 5 min of sample injection from each mouse. The collected blood was kept in the heparinized tube with ice-bathing before the measurement. The plasma was separated from blood samples using centrifugation (3000 rpm, 5 min). The plasma was diluted 5 times using ACN and then analyzed to verify the drug and prodrug concentration within the plasma using RP-HPLC.

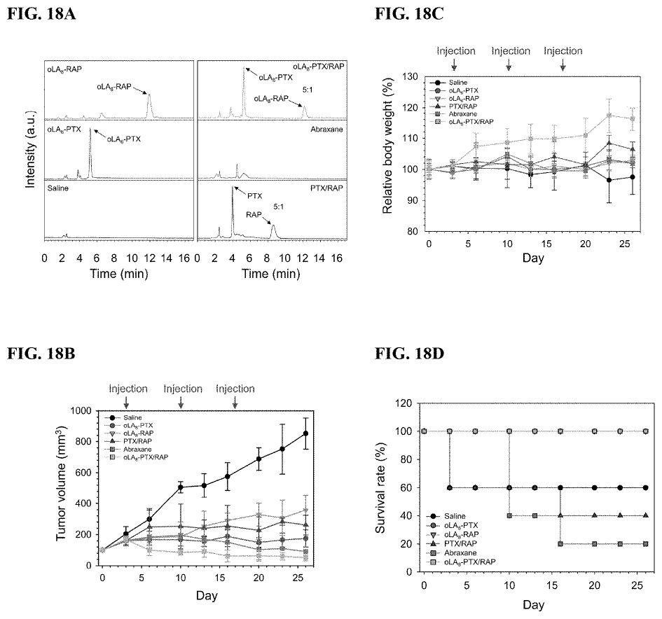

FIG. 18A shows the prodrugs and parent drugs concentration in plasma 5 min post injection. FIG. 18B displays the tumor volume over time. As seen, the prodrug combo formulation prepared using PEG-assist method outperformed both Abraxane and parent drug combination in suppressing tumor growth. The prodrug combo micellar formulation also prevented weight loss more effectively compared to other formulations (FIG. 18C). The animal injected with the prodrug combo formulation all survived over the course of study while Abraxane was found to be lethal at the same dose (FIG. 18D).

### Example 11—Lyophilization Capability of PEG-Assist Using PEG2000

The role of PEG2000 as both the solvent and lyoprotectant was confirmed by lyophilization of Example 8 formulation i.e. o(LA)₈-PTX/o(LA)₈-RAP co-loaded micelles. The micelle solutions were lyophilized for 72 hours at −35° C. The freeze-dried cakes could easily be reconstituted in water at room temperature. Following lyophilization and reconstitution, 93.3±0.8 and 93.4±0.6% of the original amounts of o(LA)₈-PTX and o(LA)₈-RAP were recovered, respectively. Particle size increased minimally from 109.1±5.6 nm to 117.7±2.5 nm after lyophilization. Negligible prodrug loss and particle size change suggest that PEG2000 acts as a suitable lyoprotectant for the lyophilization of o(LA)₈-PTX/o(LA)₈-RAP co-loaded PEG-b-PLA micelles.

### Example 12—the Effect of PEG2000 to Copolymer Ratio on Encapsulation Efficiency, Particle Size and Lyophilization Outcome

Samples in the Examples 7-8 were made again but using different amounts of PEG2000. To examine the effect of the amount of PEG2000, 10:1 and 5:1 (w/w) ratios of PEG to copolymer were used. Encapsulation efficiency and particle size before and after lyophilization were measured for all the samples. Tables 9 and 10 show the results before and after lyophilization, respectively.

TABLE 9
Encapsulation efficiency and particle size for oLA8-PTX,
oLA8-RAP or co-encapsulated oLA8-PTX/oLA8-RAP using
different amounts of PEG2000 before lyophilization

| | PEG2000 | | | | | |
|---|---|---|---|---|---|---|
| | to | % Encapsulation | | | | |
| | copolymer | efficiency | | % Drug loading | | Particle |
| | ratio | oLA8- | oLA8- | oLA8- | oLA8- | size |
| Sample | (w/w) | PTX | RAP | PTX | RAP | (nm) |
| Individual | 10:1 | 53.3 | — | 34.8 | — | 103.3 ± |
| oLA8-PTX | | | | | | 57.8 |
| Individual | 10:1 | — | 40.1 | | 28.6 | 113.1 ± |
| oLA8-RAP | | | | | | 39.9 |
| Co-loaded | 10:1 | Total: 66.1 | | Total: 39.8 | | 114.0 ± |
| oLA8-PTX/ | | | | | | 46.4 |
| oLA8-RAP | | | | | | |
| Individual | 5:1 | 36.6 | — | 26.8 | — | 122.5 ± |
| oLA8-PTX | | | | | | 81.7 |
| Individual | 5:1 | — | 45.6 | — | 31.3 | 139.4 ± |
| oLA8-RAP | | | | | | 60.6 |
| Co-loaded | 5:1 | Total: 34.8 | | Total: 25.8 | | 124.8 ± |
| oLA8-PTX/ | | | | | | 142.7 |
| oLA8-RAP | | | | | | |

TABLE 10
Encapsulation efficiency and particle size for oLA8-PTX,
oLA8-RAP or co-encapsulated oLA8-PTX/oLA8-RAP using
different amounts of PEG2000 after lyophilization

| | PEG2000 | | | Particle size |
|---|---|---|---|---|
| | to | % Encapsulation efficiency after | | after |
| | copolymer | lyophilization | | lyophilization |
| Sample | ratio | oLA8-PTX | oLA8-RAP | (nm) |
| Individual oLA8-PTX | 10:1 | 110.3 | — | 111.9 ± 49.8 |
| Individual oLA8-RAP | 10:1 | — | 76.6 | 126.2 ± 34.5 |
| Co-loaded oLA8- | 10:1 | Total: 83.6 | | 128.8 ± 46.5 |
| PTX/oLA8-RAP | | | | |
| Individual oLA8-PTX | 5:1 | 45.6 | — | 117.7 ± 49.0 |
| Individual oLA8-RAP | 5:1 | — | 57.7 | 133.1 ± 51.8 |
| Co-loaded oLA8- | 5:1 | Total: 51.2 | | 148.3 ± 62.7 |
| PTX/oLA8-RAP | | | | |

### Example 13—Encapsulation of Gefitinib-Based PROTAC3 in PEG-b-PLA Micelles

PEG-assist was used to encapsulate Gefitinib-based PROTAC3 (PROTAC) in PEG4k-b-PLA2.2k micelles. The structure of PROTAC is shown below.

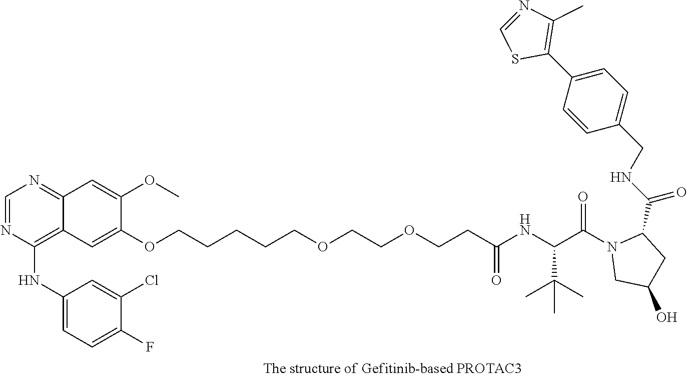 [SMILES: COc1cc2ncnc(Nc3ccc(F)c(Cl)c3)c2cc1OCCCCCOCCOCCC(=O)N[C@H](C(=O)N1C[C@H](O)C[C@H]1C(=O)NCc1ccc(-c2scnc2C)cc1)C(C)(C)C]

Briefly, 0.2 mg PROTAC, 0.8 mg PEG4k-b-PLA2.2k and 16 mg PEG1000 were mixed and heated to 60° C. and kept at this temperature for 30 min. Samples were cooled down to 40° C. and incubated for 2 h. Hydration was carried out with 200 μL DI water at the same temperature. Samples were cooled down to room temperature, centrifuged at 10000 g for 10 min and analyzed. Table 11 shows the results for encapsulation.

TABLE 11
The outcome of Encapsulation of Gefitinib-based PROTAC3
in PEG-b-PLA micelles using PEG-assist method

| Parameter | Value |
|---|---|
| Size (nm) | 34.1 ± 6.5 |
| PdI | 0.208 |
| Zeta potential (mV) | 3.39 ± 14.2 |
| % Encapsulation Efficiency | 83.0 |
| % Drug Loading | 17.2 |

### Example 14—Encapsulation of PROTAC in PSMA Targeting PEG-b-PLA Micelles

Different amounts of ACUPA-PEG-b-PLA (the structure is shown below) were added to the formulation in Example 13 to make PSMA-targeting PROTAC-loaded micelles. To the Example 13 formulation, 1.4 or 2.7 mg ACUPA-PEG5k-b-PLA2k or ACUPA-PEG3.4k-b-PLA2k were added and samples were made using the same parameters used in Example 13. Table 12 shows the results of the experiments.

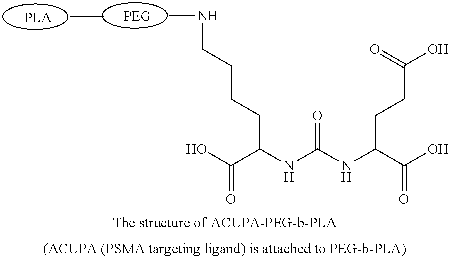

TABLE 12
The outcome of Encapsulation of PROTAC in
PSMA targeting ACUPA-PEG-b-PLA micelles

| | Amount of | | | | % | |
|---|---|---|---|---|---|---|
| | ACUPA- | | | Zeta | Encap- | % |
| | PEG-PLA | Size | | potential | sulation | Drug |
| Sample | added | (nm) | PdI | (mV) | Efficiency | Loading |
| ACUPA3.4k- | 1.4 mg | 40.3 ± 10.3 | 0.286 | −14.7 ± 15.1 | 92.6 | 11.8 |
| 1.4 | | | | | | |
| ACUPA3.4k- | 2.7 mg | 44.2 ± 21.9 | 0.219 | −18.7 ± 17.4 | 98.8 | 13.0 |
| 2.7 | | | | | | |
| ACUPA5k- | 1.4 mg | 46.4 ± 26.9 | 0.248 | −22.8 ± 14.4 | 92.9 | 14.7 |
| 1.4 | | | | | | |
| ACUPA5k- | 2.7 mg | 58.9 ± 18.8 | 0.227 | −29.5 ± 16.5 | 98.8 | 13.0 |
| 2.7 | | | | | | |

### Example 15. Encapsulation of oLA₈-DTX in PSMA Targeting ACUPA-PEG-b-PLA Micelles

oLA₈-DTX (1 mg) and PEG4k-b-PLA2.2k (1 mg) with different amounts of ACUPA-PEG5k-b-PLA2k or ACUPA-PEG3.4k-b-PLA2k ranging from 0.1-0.5 mg were dissolved in PEG2000 (20 mg) at 70° C. The samples were cooled down to 60° C. and kept for 2 h. Deionized water (1 mL, 60° C.) was added to the samples followed by vortexing. The samples were cooled down to room temperature for 30 min and analyzed. Table 13 and Table 14 show the composition of the samples and the outcome of encapsulation, respectively.

TABLE 13
Composition of different DTX prodrug encapsulated
micelles with or without ACUPA ligand

| | | PEG4k-b- | | PEG Mw of | ACUPA- |
|---|---|---|---|---|---|
| | oLA₈-DTX | PLA2.2k | PEG2000 | ACUPA-PEG- | PEG-b-PLA |
| Sample ID | (mg) | (mg) | (mg) | b-PLA (Da) | (mg) |
| No ACUPA | 1 | 1 | 20 | — | 0 |
| ACUPA3.4k-0.1 | 1 | 1 | 20 | 3400 | 0.1 |
| ACUPA3.4k-0.2 | 1 | 1 | 20 | 3400 | 0.2 |
| ACUPA3.4k-0.5 | 1 | 1 | 20 | 3400 | 0.5 |
| ACUPA5k-0.1 | 1 | 1 | 20 | 5000 | 0.1 |
| ACUPA5k-0.2 | 1 | 1 | 20 | 5000 | 0.2 |
| ACUPA5k-0.5 | 1 | 1 | 20 | 5000 | 0.5 |

TABLE 14
The outcome of Encapsulation of DTX prodrug
in PSMA targeting ACUPA-PEG-b-PLA micelles

| | | | Zeta | | |
|---|---|---|---|---|---|
| | Size | | potential | % Encapsulation | % Drug |
| Sample ID | (nm) | PdI | (mV) | efficiency | loading |
| No ACUPA | 93.2 ± 47.8 | 0.143 | 0.15 ± 10.2 | 102.7 ± 9.5 | 50.4 ± 2.3 |
| ACUPA3.4k-0.1 | 95.6 ± 47.8 | 0.144 | −9.33 ± 9.8 | 94.4 ± 4.4 | 46.0 ± 1.2 |
| ACUPA3.4k-0.2 | 91.1 ± 33.7 | 0.14 | −10.9 ± 4.9 | 103.0 ± 2.3 | 45.7 ± 5.6 |
| ACUPA3.4k-0.5 | 107.4 ± 34.3 | 0.098 | −12.8 ± 5.0 | 72.7 ± 11.6 | 32.4 ± 3.5 |
| ACUPA5k-0.1 | 95.2 ± 44.7 | 0.139 | −8.6 ± 6.3 | 84.3 ± 9.6 | 43.1 ± 2.8 |
| ACUPA5k-0.2 | 97.7 ± 43.9 | 0.196 | −9.9 ± 9.2 | 91.3 ± 18.3 | 42.8 ± 4.9 |
| ACUPA5k-0.5 | 92.4 ± 25.5 | 0.138 | −10.5 ± 7.2 | 103.2 ± 10.5 | 40.5 ± 2.5 |

### Example 16—Lyophilization of PSMA Targeting oLA₈-DTX ACUPA-PEG-b-PLA Micelles

The samples in Example 15 were lyophilized at −35° C. and 15 Pa in a VirTis Advantage Pro shelf freeze-dryer for 72 h. The samples were reconstituted using the original amount of water they contained before lyophilization and analyzed. Table 15 shows the outcome of lyophilization.

TABLE 15
Different characteristics of DTX prodrug loaded
ACUPA micelles after lyophilization

| | | | Zeta | |
|---|---|---|---|---|
| | Size | | potential | % Encapsulation |
| Sample ID | (nm) | PdI | (mV) | efficiency |
| No ACUPA | 93.2 ± 50.3 | 0.14 | −1.25 ± 3.6 | 85.4 ± 11.1 |
| ACUPA3.4k-0.1 | 109.0 ± 38.4 | 0.082 | −10.7 ± 7.5 | 98.6 ± 0.3 |
| ACUPA3.4k-0.2 | 96.2 ± 47.0 | 0.129 | −11.7 ± 6.5 | 97.5 ± 2.0 |
| ACUPA3.4k-0.5 | 130.9 ± 96.1 | 0.25 | −11.9 ± 3.3 | 92.9 ± 11.7 |
| ACUPA5k-0.1 | 102.6 ± 56.9 | 0.168 | −9.3 ± 8.5 | 92.5 ± 7.0 |
| ACUPA5k-0.2 | 110.9 ± 27.9 | 0.183 | −9.0 ± 3.0 | 89.4 ± 12.9 |
| ACUPA5k-0.5 | 120.7 ± 36.1 | 0.262 | −10.8 ± 5.0 | 89.3 ± 6.8 |

### Example 17. Stability of PSMA Targeting oLA₈-DTX ACUPA-PEG-b-PLA Micelles

The samples in Example 16, were monitored for stability after reconstitution. The particle size and PdI was measured at different time points (1, 2 and 7 days). Table 16 shows the results for stability.

TABLE 16
Particle size and PdI change over time for
DTX prodrug loaded ACUPA micelles

| | day 0 | | day 1 | | day 2 | | day 7 | |
|---|---|---|---|---|---|---|---|---|
| | Size | | Size | | Size | | Size | |
| Sample ID | (nm) | PdI | (nm) | PdI | (nm) | PdI | (nm) | PdI |
| No ACUPA | 93.2 ± | 0.143 | 95.0 ± | 0.162 | 93.8 ± | 0.131 | 94.5 ± | 0.174 |
| | 47.8 | | 34.4 | | 41.8 | | 49.8 | |
| ACUPA3.4k- | 95.6 ± | 0.144 | 97.0 ± | 0.109 | 95.0 ± | 0.172 | 97.4 ± | 0.146 |
| 0.1 | 47.8 | | 41.0 | | 37.7 | | 42.5 | |
| ACUPA3.4k- | 91.1 ± | 0.14 | 92.4 ± | 0.142 | 94.4 ± | 0.069 | 95.9 ± | 0.11 |
| 0.2 | 33.7 | | 35.0 | | 31.0 | | 36.5 | |
| ACUPA3.4k- | 107.4 ± | 0.098 | 106.3 ± | 0.101 | 107.9 ± | 0.071 | 109.0 ± | 0.148 |
| 0.5 | 34.3 | | 40.2 | | 36.9 | | 41.0 | |
| ACUPA5k- | 95.2 ± | 0.139 | 94.9 ± | 0.146 | 94.4 ± | 0.18 | 96.7 ± | 0.166 |
| 0.1 | 44.7 | | 48.4 | | 33.6 | | 34.4 | |
| ACUPA5k- | 97.7 ± | 0.196 | 100.1 ± | 0.162 | 99.3 ± | 0.155 | 105.2 ± | 0.176 |
| 0.2 | 43.9 | | 48.2 | | 39.7 | | 49.5 | |
| ACUPA5k- | 92.4 ± | 0.138 | 85.5 ± | 0.135 | 86.2 ± | 0.145 | 86.5 ± | 0.128 |
| 0.5 | 25.5 | | 26.5 | | 29.2 | | 25.3 | |

### Example 18. Encapsulation of Cerium Oxide Nanoparticles (CeNP) in PEG-b-PLA Micelles Using PEG-Assist Method

To obtain 50 or 20 wt,% target loading, 10 mg CeNP 5 nm) together with 10 or 40 mg PEG4k-b-PLA2.2k were mixed with 200 or 800 mg PEG2000, respectively, and heated to 70° C. The mixtures were cooled down to 60° C. and incubated for 2 h. 1 mL water at the same temperature was added to each mixture and vortexed in order to obtain 10 mg/mL CeNP. The samples were cooled down to room temperature and analyzed. Table 17 shows the outcome of encapsulation of CeNP using PEG-assist method.

TABLE 17
The outcome of Encapsulation of CeNP in PEG-b-PLA micelles

| | Target | | | Zeta | % Encap- | |
|---|---|---|---|---|---|---|
| | loading | Size | | potential | sulation | % |
| Sample | (wt. %) | (nm) | PdI | (mV) | Efficiency | Loading |
| CeNP-50 | 50 | 27.8 ± 10.5 | 0.175 | 10.0 ± 5.4 | 101.9 ± 4.2 | 50.6 ± 5.8 |
| CeNP-20 | 20 | 78.1 ± 130.3 | 0.560 | 35.2 ± 4.4 | 68.5 ± 12.6 | 13.7 ± 3.7 |

### Example 19—Lyophilization of CeNP Encapsulated PEG-b-PLA Micelles

The samples in Example 18 were lyophilized at −35° C. and 15 Pa in a VirTis Advantage Pro shelf freeze-dryer for 72 h. The samples were reconstituted using the original amount of water they contained before lyophilization and analyzed. Table 18 shows the outcome of lyophilization.

TABLE 18
Different characteristics of CeNP loaded
PEG-b-PLA micelles after lyophilization

| | | | Zeta potential | % Encapsulation |
|---|---|---|---|---|
| Sample | Size (nm) | PdI | (mV) | Efficiency |
| CeNP-50 | 25.3 ± 10.4 | 0.243 | 10.5 ± 8.4 | 94.4 ± 17.8 |
| CeNP-20 | 105.8 ± 118.8 | 0.429 | 31.7 ± 9.5 | 56.0 ± 19.6 |

### EQUIVALENTS

While certain embodiments have been illustrated and described, a person with ordinary skill in the art, after reading the foregoing specification, can effect changes, substitutions of equivalents and other types of alterations to the conjugates and micelles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

What is claimed is:

1. A method of manufacture comprising:
preparing at a first temperature a non-supersaturated solution comprising a micelle-forming block copolymer and a drug in a polyethylene glycol (PEG) solvent; and
diluting the non-supersaturated solution with water at a second temperature to form an aqueous solution comprising drug-loaded micelles without crystallization of the PEG block(s) of the block copolymer;
wherein
the block copolymer comprises at least one PEG block and a second polymer block that is not PEG,
the drug has a water solubility of about or less than 10 mg/mL at 25° C., and
the second temperature is lower than the first temperature, but does not allow the cooled solution to become supersaturated.

2. The method of claim 1, wherein preparing the non-supersaturated solution comprises heating a mixture of the block copolymer, drug and the PEG solvent at the first temperature until the non-supersaturated solution forms.

3. The method of claim 1, wherein preparing the non-supersaturated solution comprises i) heating a mixture of the block copolymer and the PEG solvent to form a solution or partial solution, adding the drug, and heating at the first temperature the mixture of block copolymer, drug, and PEG solvent to form the non-supersaturated solution; or ii) heating a mixture of the drug and the PEG solvent to form a solution or partial solution, adding the block copolymer, and heating at the first temperature the mixture of drug, block copolymer, and PEG solvent to form the non-supersaturated solution.

4. The method of claim 1, wherein the non-supersaturated solution is equilibrated at the first temperature for at least 0.1 h after formation of the non-supersaturated solution.

5. The method of claim 1, wherein diluting the non-supersaturated solution with water is carried out by mixing to homogeneity in less than an hour.

6. The method of claim 1, wherein diluting the non-supersaturated solution with water is carried out by a single addition of water or two or more additions of water to the non-supersaturated solution.

7. The method of claim 1, wherein the non-supersaturated solution is prepared continuously and diluted continuously with water to continuously form the aqueous solution.

8. The method of claim 1, wherein the non-saturated solution is an under-saturated solution at the first temperature.

9. The method of claim 1, wherein the first temperature ranges from 40° C. to 90° C.

10. The method of claim 1, wherein the non-supersaturated solution is a saturated solution at the second temperature.

11. The method of claim 1, wherein the second temperature ranges from 30° C. to 50° C.

12. The method of claim 1 further comprising i) cooling the non-supersaturated solution to the second temperature such that the solution remains non-supersaturated; and/or ii) freeze-drying the aqueous solution comprising drug-loaded micelles to provide a freeze-dried powder.

13. The method of claim 1, wherein the PEG is not substantially removed from the aqueous solution.

14. The method of claim 1, wherein the eutectic point of the aqueous solution is above −50° C.

15. The method of claim 1, wherein the aqueous solution is not dialyzed.

16. The method of claim 1, wherein diluting the non-supersaturated solution with water induces liquid-liquid phase separation between the block copolymer, water and the PEG solvent.

17. The method of claim 1, wherein the PEG solvent has a weight average molecular weight of about 800 Da about 4 kDa.

18. The method of claim 1, wherein the non-saturated solution comprises 0.1 wt % to 50 wt % block copolymer.

19. The method of claim 1, wherein the non-saturated solution comprises 0.005 wt % to 50 wt % drug.

20. The method of claim 1, wherein the block copolymer is PEG-b-PLA, PEG-b-PLA-b-PEG, PEG-b-PLA, PEG-b-PLA-b-PEG, PEG-b-PPG-b-PEG, PEG-PS, or PEG-PMMA.

21. The method of claim 20, wherein the molecular weight of the poly(ethylene glycol) block of PEG-b-PLA is about 1,000 to about 35,000 g/mol and the molecular weight of the acid) block of PEG-b-PLA is about 1,000 to about 15,000 g/mol.

22. The method of claim 1, wherein the block copolymer is poly(ethylene glycol)-block-polylactic acid (PEG-b-PLA).

23. The method of claim 1, wherein the PEG block of the block copolymer is terminated in a targeting ligand.

24. The method of claim 1, wherein the drug is selected from the group consisting of paclitaxel, docetaxel, carbazitaxel, rapamycin, everolimus, selumetinib, binimetinib, GDC-0623, ICU 189150, doxorubicin, etoposide, 17-AAG, bicalutamide, embelin, suberoylanilide hydroxamic acid, β-lapachone, pifithrin-μ, sagopilone, thiocoraline, ABT-263, podophyllotoxin, simvastatin, efavirenz, platin-based prodrugs, VE-822, AZD5363, teniposide, AZD8055, rutin, PROTAC, cerium oxide nanoparticles, derivatives and/or conjugates of any of the foregoing, and combinations of two or more thereof.

25. The method of claim 1, wherein the drug is an oligolactic acid conjugate.
